(19) United States
(12) Patent Application Publication
(10) Pub. No.: US 2024/0306643 A1
(43) Pub. Date: Sep. 19, 2024

(54) ALKOXIDE-BASED SOLIDIFICATION USING CONCENTRATED ALKALI METAL HYDROXIDES

(71) Applicant: ECOLAB USA INC., Saint Paul, MN (US)

(72) Inventors: Victor Fuk-Pong Man, Saint Paul, MN (US); Gang Pu, Saint Paul, MN (US); Mark Schomers, Saint Paul, MN (US)

(21) Appl. No.: 18/605,976

(22) Filed: Mar. 15, 2024

Related U.S. Application Data
(60) Provisional application No. 63/490,815, filed on Mar. 17, 2023.

(51) Int. Cl. A01N 59/00; A01N 25/12; A01P 1/00; A61L 2/18; A61L 101/02; C11D 1/72; C11D 3/04; C11D 3/20; C11D 3/33; C11D 3/48

(52) U.S. Cl. CPC A01N 59/00; A01N 25/12; A61L 2/18; C11D 1/72; C11D 3/044; C11D 3/2044; C11D 3/33; C11D 3/48; A01P 1/00; A61L 2101/02

(57) ABSTRACT

Solid compositions having a caustic source and polyol with a caustic:water weight ratio in the solid composition between 60:40 to less than about 70:30 are provided. Methods of making the solid compositions using a concentrated alkali metal hydroxide beneficially reduces or eliminates the use of solid caustic beads commonly used to make solid caustic compositions for use in cleaning, sanitizing, and disinfecting.

### CROSS REFERENCE TO RELATED APPLICATIONS

This application claims priority under 35 U.S.C. § 119 to provisional application Ser. No. 63/490,815, filed Mar. 17, 2023, which is herein incorporated by reference in its entirety.

### TECHNICAL FIELD

The present disclosure relates to the field of solid compositions for various applications of use. Solid compositions having a caustic source and polyol with a caustic:water weight ratio in the solid composition between 60:40 to less than about 70:30 are provided. Methods of making the solid compositions using a concentrated alkali metal hydroxide beneficially reduces or eliminates the use of solid caustic beads commonly used to make solid caustic compositions for use in cleaning, sanitizing, and disinfecting.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Alkali metal hydroxides, commonly referred to as caustic, is commonly sold in solid form (pellets, flakes, blocks) and is frequently used in manufacturing processes. The manufacturing of caustic beads is energy intensive and compositions containing caustic beads are generally hygroscopic. Additionally, there are safety concerns surrounding the transportation and handling of other strong bases, such as alkoxides. Alkoxides are strong bases which readily decompose tissues and proteins when contacted and further are unstable in water, making storage and transportation difficult in the certain weather conditions or in the event moisture is present in the storage container.

Despite these challenges of using solid caustic and alkoxides there remains advantages to using solid compositions, such as caustic cleaning compositions. For example, storing and transporting of solid concentrates are less expensive than the storage and transporting of liquids. There are also fewer safety and stability challenges associated with transporting and handling of solid compositions.

Therefore, it is an object of the disclosure to provide novel solid compositions and methods of making the solid compositions which provide cost effective alternatives to the purchase of solid caustic to incorporate into a solid composition.

Accordingly, it is an objective of the claimed disclosure to develop solid compositions that provide a contiguous solid suitable for use in various solid compositions.

It is an objective to develop solid compositions that can be pre-formed and used in various solidification processes (e.g. pressed, cast, extruded) and/or can be used to form a solid in situ or at a point of use.

It is a further objective to develop solid compositions that minimize the use of solid caustic beads required to make caustic solid compositions.

It is a further object of the disclosure to provide methods for converting caustic liquid into an alkoxide for use in various solid compositions. Methods can include in situ generation or preforming solid complexes (e.g. dehydrated binding agents) to be used in forming a solid composition.

Other objects, embodiments and advantages of this disclosure will be apparent to one skilled in the art in view of the following disclosure, the drawings, and the appended claims.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

In embodiments, solid compositions comprising an alkali metal hydroxide, a polyol, and water are provided, wherein the solid has a weight ratio of alkali metal hydroxide to water from about 60:40 to less than about 70:30, wherein the water is from any component of the solid composition and/or water added as a raw material, and wherein the solid is a contiguous solid, powder or granule.

In further embodiments, methods of making a solid composition comprise: combining a concentrated alkali metal hydroxide and a polyol to generate in situ a solid composition, wherein the concentrated alkali metal hydroxide comprises greater than 50% (actives basis) liquid alkali metal hydroxide, and wherein the solid has a weight ratio of alkali metal hydroxide to water from about 60:40 to less than about 70:30, wherein the water is from any component of the solid composition and/or water added as a raw material, and wherein the solid is a cast solid, tablet, block, or powder, and wherein the solid is a dimensionally stable solid or a flowable powder.

In further embodiments, methods of using the solid compositions comprise: generating a use solution of the solid compositions described herein; contacting an article or surface in need of cleaning, disinfecting, and/or sanitizing with the use solution; and cleaning, disinfecting, and/or sanitizing the article or surface.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

FIG. 1 shows a graph of traditional caustic-based cast solids reliance on caustic to water ratio for solidification to form caustic monohydrate and corresponding melting points. Which melts at 66° Celsius as shown in FIG. 1 depiction of varying caustic to water ratios an

FIG. 2 shows a graph of melting point of sodium hydroxide at about 145° F.

FIG. 3 shows a graph comparing dispensing rate (slope of graphs showing mass over time, y-axis and x-axis, respectively) at 100° F. of solid caustic compositions compared with inline caustic detergent as described in Example 5.

FIG. 4 shows a graph comparing dispensing rate (slope of graphs showing mass over time, y-axis and x-axis, respectively) at 140° F. of solid caustic compositions compared with inline caustic detergent as described in Example 5.

FIG. 5 shows a graph testing Composition 2-1 for dimensional stability of the solid as described in Example 6.

FIG. 6 shows a graph testing Composition 2-1 for hardness of the solid using penetrometer readings as described in Example 6.

FIG. 7 shows a graph testing Composition 2-2 for dimensional stability of the solid as described in Example 6.

FIG. 8 shows a graph testing Composition 2-2 for hardness of the solid using penetrometer readings as described in Example 6.

FIG. 9 shows a graph testing Composition 2-3 for dimensional stability of the solid as described in Example 6.

FIG. 10 shows a graph testing Composition 2-3 for hardness of the solid using penetrometer readings as described in Example 6.

Various embodiments of the present disclosure will be described in detail with reference to the drawings, wherein like reference numerals represent like parts throughout the several views. Reference to various embodiments does not limit the scope of the disclosure. Figures represented herein are not limitations to the various embodiments according to the disclosure and are presented for exemplary illustration of the invention. An artisan of ordinary skill in the art need not view, within isolated figure(s), the near infinite number of distinct permutations of features described in the following detailed description to facilitate an understanding of the present invention.

### DETAILED DESCRIPTION

The embodiments of this disclosure are not limited to particular solid compositions containing the alkoxide binding agents, methods of making and/or methods of employing the same for hard surface sanitizing and disinfecting, including antimicrobial and/or sanitizing application for cleaning compositions, along with alternative cleaning and uses for cleaning compositions, which can vary and are understood by skilled artisans. So that the disclosure may be more readily understood, certain terms are first defined. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions. This applies regardless of the breadth of the range.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

Unless defined otherwise, all technical and scientific terms used above have the same meaning as commonly understood by one of ordinary skill in the art to which embodiments of the present disclosure pertain.

The term “about,” as used herein, refers to variation in the numerical quantity that can occur, for example, through typical measuring techniques and equipment, with respect to any quantifiable variable, including, but not limited to, concentration, mass, volume, time, molecular weight, temperature, pH, humidity, molar ratios, log count of bacteria or viruses, and the like. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts.

As used herein, the term “alkoxide” refers to a conjugate base of an organic molecule having one or more hydroxyl groups and can be formed through the deprontonation of the hydroxyl group(s), which is a weak acid/base reaction. Alkoxides can be formed through the reaction of an alkali metal hydroxide and an organic molecule having one or more hydroxyl-groups or an alkylene carbonate as disclosed in U.S. patent application Ser. No. 18/605,976, claiming priority to U.S. Ser. No. 63/490,838, filed simultaneously herewith and titled “Alkoxide-Based Solidification Via Control of Reaction Equilibrium and Kinetics”, which is incorporated by reference in its entirety.

As used herein, the term “alkyl” or “alkyl groups” refers to saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

As used herein, the term “antimicrobial” refers to a compound or composition that reduces and/or inactivates a microbial population, including, but not limited to bacteria, viruses, fungi, and algae within about 10 minutes or less, about 8 minutes or less, about 5 minutes or less, about 3 minutes or less, about 2 minutes or less, about 1 minute or less, or about 30 seconds or less. Preferably, the term antimicrobial refers to a composition that provides at least about a 3-log, 3.5 log, 4 log, 4.5 log, or 5 log reduction of a microbial population in about 10 minutes or less, about 8 minutes or less, about 5 minutes or less, about 3 minutes or less, about 2 minutes or less, about 1 minute or less, or about 30 seconds or less.

As used herein, the term “cleaning” refers to a method used to facilitate or aid in soil removal, bleaching, microbial population reduction, and any combination thereof. As used herein, the term “microorganism” refers to any noncellular or unicellular (including colonial) organism. Microorganisms include all prokaryotes. Microorganisms include bacteria (including cyanobacteria), spores, lichens, fungi, protozoa, virinos, viroids, viruses, phages, and some algae. As used herein, the term “microbe” is synonymous with microorganism. For the purpose of this patent application, successful microbial reduction is achieved when the microbial populations are reduced by at least about 50%, or by significantly more than is achieved by a wash with water. Larger reductions in microbial population provide greater levels of protection.

As used herein, the term “disinfectant” refers to an agent that kills all vegetative cells including most recognized pathogenic microorganisms, using the procedure described in A.O.A.C. Use Dilution Methods, Official Methods of Analysis of the Association of Official Analytical Chemists, paragraph 955.14 and applicable sections, 15th Edition, 1990 (EPA Guideline 91-2). According to this reference a disinfectant should provide a 99.999% reduction (5-log order reduction) within 30 seconds at room temperature, 25±2° C., against several test organisms. According to embodiments of the disclosure, a disinfecting composition provides a 99.999% reduction (5-log order reduction) of the desired organisms (including bacterial contaminants) at a use temperature. Further, a disinfectant should provide a 99.99% reduction (4-log order reduction) within 30 seconds at room temperature, 25±2° C., against several test organisms. According to embodiments of the disclosure, a disinfecting composition provides a 99.99% reduction (4-log order reduction) of the desired organisms (including bacterial contaminants) at a use temperature. Further, a disinfectant should provide a 99.9% reduction (3-log order reduction) within 30 seconds at room temperature, 25±2° C., against several test organisms. According to embodiments of the disclosure, a disinfecting composition provides a 99.9% reduction (3-log order reduction) of the desired organisms (including bacterial contaminants) at a use temperature. As used herein, the term “high level disinfection” or “high level disinfectant” refers to a compound or composition that kills substantially all organisms, except high levels of bacterial spores, and is effected with a chemical germicide cleared for marketing as a sterilant by the Food and Drug Administration. As used herein, the term “intermediate-level disinfection” or “intermediate level disinfectant” refers to a compound or composition that kills mycobacteria, most viruses, and bacteria with a chemical germicide registered as a tuberculocide by the Environmental Protection Agency (EPA). As used herein, the term “low-level disinfection” or “low level disinfectant” refers to a compound or composition that kills some viruses and bacteria with a chemical germicide registered as a hospital disinfectant by the EPA. The term or abbreviation “EDTA 4Na+” refers to ethylenediaminetetraacetic acid, tetrasodium salt.

As used herein, the term “exemplary” refers to an example, an instance, or an illustration, and does not indicate a most preferred embodiment unless otherwise stated.

As used herein, the phrase “food processing surface” refers to a surface of a tool, a machine, equipment, a structure, a building, or the like that is employed as part of a food processing, preparation, or storage activity. Examples of food processing surfaces include surfaces of food processing or preparation equipment (e.g., slicing, canning, or transport equipment, including flumes), of food processing wares (e.g., utensils, dishware, wash ware, and bar glasses), and of floors, walls, or fixtures of structures in which food processing occurs. Food processing surfaces are found and employed in food anti-spoilage air circulation systems, aseptic packaging sanitizing, food refrigeration and cooler cleaners and sanitizers, ware washing sanitizing, blancher cleaning and sanitizing, food packaging materials, cutting board additives, third-sink sanitizing, beverage chillers and warmers, meat chilling or scalding waters, autodish sanitizers, sanitizing gels, cooling towers, food processing antimicrobial garment sprays, and non-to-low-aqueous food preparation lubricants, oils, and rinse additives.

As used herein, the phrase “food product” includes any food substance that might require treatment with an antimicrobial agent or composition and that is edible with or without further preparation. Food products include meat (e.g. red meat and pork), seafood, poultry, produce (e.g., fruits and vegetables), eggs, living eggs, egg products, ready to eat food, wheat, seeds, roots, tubers, leaves, stems, corns, flowers, sprouts, seasonings, or a combination thereof. The term “produce” refers to food products such as fruits and vegetables and plants or plant-derived materials that are typically sold uncooked and, often, unpackaged, and that can sometimes be eaten raw.

The term “generally” encompasses both “about” and “substantially.”

The term “hard surface” refers to a solid, substantially non-flexible surface such as a countertop, tile, floor, wall, panel, window, plumbing fixture, kitchen and bathroom furniture, appliance, engine, circuit board, and dish. Hard surfaces may include for example, health care surfaces and food processing surfaces.

As used herein, the phrase “health care surface” refers to a surface of an instrument, a device, a cart, a cage, furniture, a structure, a building, or the like that is employed as part of a health care activity. Examples of health care surfaces include surfaces of medical or dental instruments, of medical or dental devices, of electronic apparatus employed for monitoring patient health, and of floors, walls, or fixtures of structures in which health care occurs. Health care surfaces are found in hospital, surgical, infirmity, birthing, mortuary, and clinical diagnosis rooms. These surfaces can be those typified as “hard surfaces” (such as walls, floors, bed-pans, etc.), or fabric surfaces, e.g., knit, woven, and non-woven surfaces (such as surgical garments, draperies, bed linens, bandages, etc.,), or patient-care equipment (such as respirators, diagnostic equipment, shunts, body scopes, wheel chairs, beds, etc.,), or surgical and diagnostic equipment. Health care surfaces include articles and surfaces employed in animal health care.

As used herein, the term “instrument” refers to the various medical or dental instruments or devices that can benefit from cleaning with a composition according to the present disclosure.

The terms “invention” or “present invention” are not intended to refer to any single embodiment of the particular invention but encompass all possible embodiments as described in the specification and the claims.

As used herein, the phrases “medical instrument,” “dental instrument,” “medical device,” “dental device,” “medical equipment,” or “dental equipment” refer to instruments, devices, tools, appliances, apparatus, and equipment used in medicine or dentistry. Such instruments, devices, and equipment can be cold sterilized, soaked or washed and then heat sterilized, or otherwise benefit from cleaning in a composition of the present disclosure. These various instruments, devices and equipment include, but are not limited to: diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressors, trocars, dilators, cages, glassware, tubing, catheters, cannulas, plugs, stents, scopes (e.g., endoscopes, stethoscopes, and arthroscopes) and related equipment, and the like, or combinations thereof.

As used herein, the term “microbe” is synonymous with microorganism. For the purpose of this patent application, successful microbial reduction is achieved when the microbial populations are reduced by at least about 50%, or by significantly more than is achieved by a wash with water. Larger reductions in microbial population provide greater levels of protection. Differentiation of antimicrobial “-cidal” or “-static” activity, the definitions which describe the degree of efficacy, and the official laboratory protocols for measuring this efficacy are considerations for understanding the relevance of antimicrobial agents and compositions. Cleaning compositions can affect two kinds of microbial cell damage. The first is a lethal, irreversible action resulting in complete microbial cell destruction or incapacitation. The second type of cell damage is reversible, such that if the organism is rendered free of the agent, it can again multiply. The former is termed microbiocidal and the later, microbiostatic. A sanitizer and a disinfectant are, by definition, agents which provide antimicrobial or microbiocidal activity. In contrast, a preservative is generally described as an inhibitor or microbiostatic composition.

As used herein, the term “microorganism” refers to any noncellular or unicellular (including colonial) organism. Microorganisms include all prokaryotes. Microorganisms include bacteria (including cyanobacteria), spores, lichens, fungi, protozoa, virinos, viroids, viruses, phages, and some algae.

As used herein the term “polymer” refers to a molecular complex comprised of a more than ten monomeric units and generally includes, but is not limited to, homopolymers, copolymers, such as for example, block, graft, random and alternating copolymers, terpolymers, and higher “x”mers, further including their analogs, derivatives, combinations, and blends thereof. Furthermore, unless otherwise specifically limited, the term “polymer” shall include all possible isomeric configurations of the molecule, including, but are not limited to isotactic, syndiotactic and random symmetries, and combinations thereof. Furthermore, unless otherwise specifically limited, the term “polymer” shall include all possible geometrical configurations of the molecule.

As used herein, the term “sanitizer” refers to an agent that reduces the number of bacterial contaminants to safe levels as judged by public health requirements. In an embodiment, sanitizers for use in this disclosure will provide at least a 99.999% reduction (5-log order reduction). These reductions can be evaluated using a procedure set out in Germicidal and Detergent Sanitizing Action of Disinfectants, Official Methods of Analysis of the Association of Official Analytical Chemists, paragraph 960.09 and applicable sections, 15th Edition, 1990 (EPA Guideline 91-2). According to this reference a sanitizer should provide a 99.999% reduction (5-log order reduction) within 30 seconds at room temperature, 25±2° C., against several test organisms. According to embodiments of the disclosure, a sanitizing composition provides a 99.999% reduction (5-log order reduction) of the desired organisms (including bacterial contaminants) at a use temperature. Further, a sanitizer should provide a 99.99% reduction (4-log order reduction) within 30 seconds at room temperature, 25±2° C., against several test organisms. According to embodiments of the disclosure, a sanitizing composition provides a 99.99% reduction (4-log order reduction) of the desired organisms (including bacterial contaminants) at a use temperature. Further, a sanitizer should provide a 99.9% reduction (3-log order reduction) within 30 seconds at room temperature, 25±2° C., against several test organisms. According to embodiments of the disclosure, a sanitizing composition provides a 99.9% reduction (3-log order reduction) of the desired organisms (including bacterial contaminants) at a use temperature.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

As used herein, the term “substantially free” refers to compositions completely lacking the component or having such a small amount of the component that the component does not affect the effectiveness of the composition. The component may be present as an impurity or as a contaminant and shall be less than 0.5 wt. %. In another embodiment, the amount of the component is less than 0.1 wt. % and in yet another embodiment, the amount of component is less than 0.01 wt. %.

The term “surfactant” as used herein is a compound that contains a lipophilic segment and a hydrophilic segment, which when added to water or solvents, reduces the surface tension of the system.

As used herein, the term “ware” refers to items such as eating and cooking utensils, dishes, and other hard surfaces such as showers, sinks, toilets, bathtubs, countertops, windows, mirrors, transportation vehicles, and floors. As used herein, the term “ware washing” refers to washing, cleaning, or rinsing ware. Ware also refers to items made of plastic. Types of plastics that can be cleaned with the compositions according to the disclosure include but are not limited to, those that include polycarbonate polymers (PC), acrilonitrile-butadiene-styrene polymers (ABS), and polysulfone polymers (PS). Another exemplary plastic that can be cleaned using the compounds and compositions of the disclosure include polyethylene terephthalate (PET).

As used herein, “weight percent,” “wt.-%,” “percent by weight,” “ % by weight,” and variations thereof refer to the concentration of a substance as the weight of that substance divided by the total weight of the composition and multiplied by 100. It is understood that, as used here, “percent,” “ %,” and the like are intended to be synonymous with “weight percent,” “wt.-%,” etc.

The solid compositions, methods of making the compositions, and methods of use of the present disclosure may comprise, consist essentially of, or consist of the components and ingredients of the present disclosure as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

### Solid Compositions

Solid compositions described herein include an alkali metal hydroxide, a polyol, and water, wherein the solid has a weight ratio of alkali metal hydroxide to water from about 60:40 to less than about 70:30, wherein the water is from any component of the solid composition and/or water added as a raw material, and wherein the solid is a contiguous solid, powder or granule. In embodiments the weight ratio of alkali metal hydroxide to water in the solid is from about 60:40 to less than about 70:30, from about 60:40 to about 69:31, or preferably from about 60:40 to less than about 65:35. In these compositions, the caustic:water ratio refers to the ratio in the solid composition, the finished good ratio after reaction, for example from neutralization of acidic components.

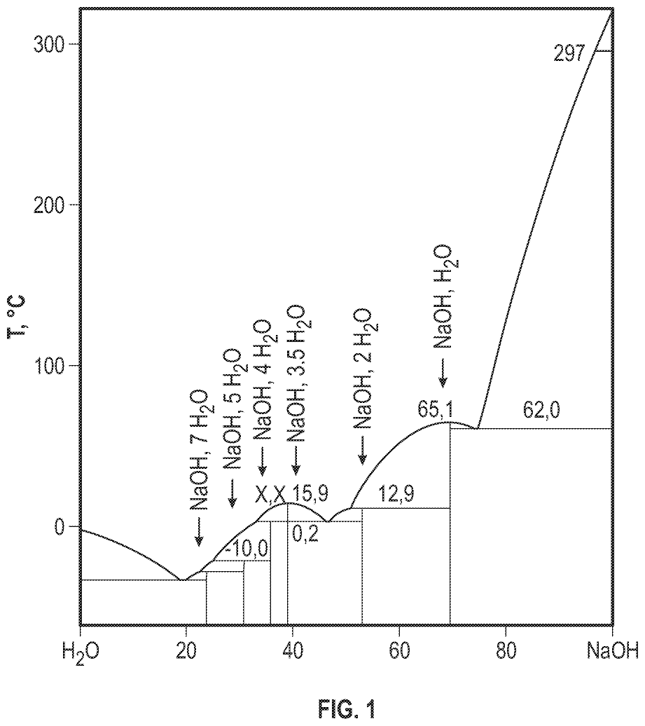

Traditional caustic-based cast solids rely on a caustic to water ratio of 69:31 for complete solidification to form caustic monohydrate which melts at 66º Celsius as shown in FIG. 1 depiction of varying caustic to water ratios and corresponding melting points. When the ratio is below the traditional 69:31 caustic:water, an incomplete solid is formed with lower melting temperature and therefore results in too fast of dissolution. In the described embodiments, the solid composition lowers the ratio of alkali metal hydroxide to water in the solid while still beneficially providing a complete solid with higher melting temperature and slower dissolution via a reaction of caustic with a reagent. As one skilled in the art will ascertain, the solid composition can vary in characteristics quantified by hardness measurements (as described in Examples using a penetrometer) or dimensional stability (as described in the Examples assessing changes in size of the solids), as well as various visual observations with respect to weeping and/or dryness of the exterior of the solid composition. These quantifiable and observed solid characteristics beneficially allow the solid compositions to be used in various applications from solid cast products to in-situ generation of solids, or forming powders and granules.

The compositions are able to provide solids at a reduced caustic:water weight ratio in the solid (i.e. after solidification), The caustic:water weight ratios are calculated based on the total components combined to form a solid composition and the resulting water in the solid composition. For example, each component is evaluated if it contains any water or caustic to calculate the weight sum of these values. The NaOH:H₂O ratio is then calculated by

$$\frac{caustic \%}{( caustic \% + water \% )} .$$

The amount of caustic neutralized by any acidic polymers is also calculated to determine the final caustic:water weight ratios in the solid compositions, resulting in the weight ratio being active caustic (after neutralization) to water (from all components). It is noted that only the two components are included in this calculation, however it does not indicate that there are not other components in the solid composition. For example, a 70:30 caustic:water weight ratio does not indicate that the solid composition has 70 wt-% active caustic, as other components may be formulated in the solid as well.

Although this caustic: water weight ratios in the solid composition can be calculated the ratio can also be analytically confirmed by an acid/base titration using HCl that determines active alkalinity along with analytical calculation for water content, such as with the Karl Fischer method or TGA (Thermal Gravimetric Analysis). The Karl Fischer method is less preferred for higher alkaline/caustic compositions where instead differential scanning calorimetry (DSC) scans are preferred.

The solid compositions are contiguous, meaning that the solid hydrates that make up the solid composition where the E-form hydrates are adjacent to one another in the solid. The contiguous solid composition forms as a result of the alkoxide·alkali·nH₂O sharing water molecule throughout the solid composition. This is described in simplified terms with the alkali metal hydroxidate·nH₂O in the solid compositions.

Without being limited according to a particular mechanism of action, the methods of making the solid compositions are different from conventional caustic solids containing both solid caustic (e.g. beads) and/or caustic with an ash source along with a solid alkoxide as there is no solidification of both alkalinity sources into hydrate solids. The disclosed compositions and methods provides solidification of caustic (and optionally carbonate as well) can be achieved simultaneously and in the same contiguous solid composition. These differ from conventional caustic solids in having a weight ratio of caustic:water in the formed solid that is lower, including below about 70:30, meaning that more caustic and less water is to be included for obtaining a solid composition. The compositions herein having less caustic and more water in the weight ratio provide solids without the solid becoming too softened or losing its shape and beneficially permit the addition of performance and process enhanced in the solid composition formulations.

In embodiments the solid compositions are dimensionally stable. The solid compositions are considered to exhibit dimensional stability if the measured dimensions as dependent upon the shape of the solid (e.g. combined diameter and height) swelling or growth is less than approximately 3%, and particularly less than approximately 2%. The time and temperature conditions for the measurement of dimensional stability can be at 4 weeks in an oven, wherein the oven is an environmental chamber between about 40° C.-50° C. and about 65% relative humidity.

The solid compositions comprise the alkali metal hydroxide, polyol, and water. In embodiments at least one additional functional ingredient is included, including for example surfactants, chelants, or additional alkaline carriers. Exemplary ranges of the reagents to make the solid compositions according to the disclosure are shown in Tables 1A-1D each in weight percentage.

TABLE 1A

| | First Exemplary | Second Exemplary | Third Exemplary |
|---|---|---|---|
| Material | Range wt.-% | Range wt.-% | Range wt.-% |
| Alkali metal | 10-99 | 20-90 | 50-90 |
| hydroxide | | | |
| Polyol | 1-20 | 1-15 | 1-10 |
| Additional | 0-80 | 2-60 | 5-50 |
| Functional | | | |
| Ingredients | | | |
| Total | 100 | 100 | 100 |

TABLE 1B

| | First Exemplary | Second Exemplary | Third Exemplary |
|---|---|---|---|
| Material | Range wt.-% | Range wt.-% | Range wt.-% |
| Alkali metal | 10-99 | 20-90 | 50-90 |
| hydroxide | | | |
| Polyol | 1-20 | 1-15 | 1-10 |
| Surfactant(s) | 0-40 | 1-20 | 1-10 |
| Additional | 0-80 | 2-60 | 5-50 |
| Functional | | | |
| Ingredients | | | |
| Total | 100 | 100 | 100 |

TABLE 1C

| | First Exemplary | Second Exemplary | Third Exemplary |
|---|---|---|---|
| Material | Range wt.-% | Range wt.-% | Range wt.-% |
| Alkali metal | 10-99 | 20-90 | 50-90 |
| hydroxide | | | |
| Polyol | 1-20 | 1-15 | 1-10 |
| Surfactant(s) | 0-40 | 1-20 | 1-10 |
| Additional | 0-60 | 1-50 | 1-50 |
| alkaline | | | |
| carrier | | | |
| Additional | 0-80 | 2-60 | 5-50 |
| Functional | | | |
| Ingredients | | | |
| Total | 100 | 100 | 100 |

TABLE 1D

| | First Exemplary | Second Exemplary | Third Exemplary |
|---|---|---|---|
| Material | Range wt.-% | Range wt.-% | Range wt.-% |
| Alkali metal | 10-99 | 20-90 | 50-90 |
| hydroxide | | | |
| Polyol | 1-20 | 1-15 | 1-10 |
| Surfactant(s) | 0-40 | 1-20 | 1-10 |
| Chelant | 1-30 | 1-20 | 5-20 |
| Additional | 0-80 | 2-60 | 5-50 |
| Functional | | | |
| Ingredients | | | |
| Total | 100 | 100 | 100 |

In an embodiment, the compositions have at least between about 50%and about 100% reduction, at least about 50%, at least about 60%, at least about 70%, at least about 80%, or at least about 90% reduction in solid caustic (e.g. caustic beads). In some embodiments, the solid compositions include alkali metal hydroxide wherein less than about 40 wt-%, less than about 30 wt-%, less than about 20 wt-%, less than about 10 wt-%, or 0 wt-% of the alkali metal hydroxide is from a solid caustic bead.

### Alkali Metal Hydroxide

The solid compositions contain at least one alkali metal hydroxide as a caustic source. As referred to herein, caustic is synonymous to hydroxide. In an embodiment of the disclosure, any suitable source of caustic may be used. In an embodiment, an alkali metal caustic source may be used. For example, caustic sources may be in the form of sodium hydroxide, potassium hydroxide, lithium hydroxide, derivatives thereof, or and combinations thereof. An example of a derivative of a caustic source is a preformed alkoxide.

In the methods of making the solid composition the alkali metal hydroxide is a solution or a liquid alkali metal hydroxide. In further embodiments additional caustic source can be included in the form of a solid, such as caustic beads, pellets, flakes, powder, granules, and the like may be combined with the liquid alkali metal hydroxide.

In an embodiment a higher active caustic liquid is preferred for the control of equilibrium reaction and kinetics for the generation of solid compositions. In an embodiment, the molar ratio of caustic to reagent (e.g. propylene glycol) is about 1:1 to about 10:1 molar ratio, about 1:1 to about 8:1 molar ratio, about 1:1 to about 6:1 molar ratio, and preferably about 1:1. In exemplary embodiments of the Examples, the reaction of glycol reagents is faster to produce the solid compositions.

In an embodiment, a concentrated caustic can be used in the methods of making the solid. In an embodiment, 70% NaOH is preferred over a 50% NaOH to provide the 1:1 (or greater) molar ratio of caustic to reagent. In preferred embodiments, a concentrated alkali metal hydroxide comprises greater than 50% (actives basis) liquid alkali metal hydroxide. In some embodiments, the concentrated alkali metal hydroxide is from about 69% to about 74% (actives basis) liquid alkali metal hydroxide, preferably from about 70% to about 73% (actives basis) liquid alkali metal hydroxide. The concentrated alkali metal hydroxide is maintained a sufficiently high temperatures to prevent premature solidification. In an embodiment the concentrated alkali metal hydroxide is maintained, handled or otherwise processed at a temperature of at least about 66° C., or from about 66° C. to about 85° C.

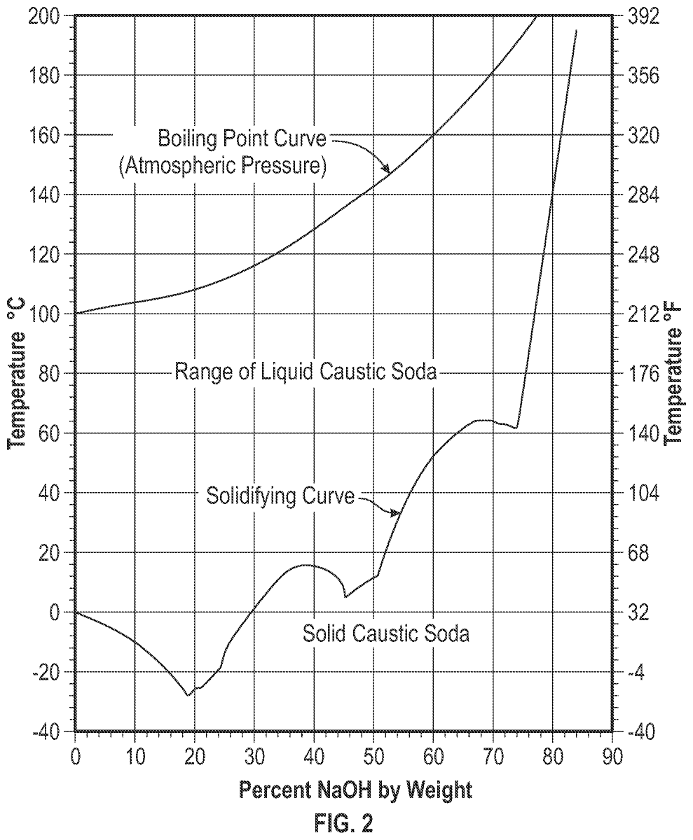

In embodiments the concentrated alkali metal hydroxide can include an in-situ concentrated molten caustic. For example, a method of making the solid compositions can include an initial step of concentrating an alkali metal hydroxide. In an embodiment, concentrating can include concentrating a liquid alkali metal hydroxide having 50% (actives basis) or less alkali metal hydroxide by evaporation. Evaporation includes any process of drying the caustic, including by heat. An exemplary process for evaporation can include a continuous, multi-effect evaporation to concentrate the caustic to about 69% to about 74% based on the caustic melting (freezing) point around 145° F. as depicted in FIG. 2. As shown in FIG. 2, sodium hydroxide is in liquid form below 74% at approximately 145° F. and increasing above this wt % the sodium hydroxide requires much higher temperatures to melt it from the solid form.

In alternative embodiments the concentrated alkali metal hydroxide can include using a mixture of caustic beads and caustic liquid, wherein the methods of making the solid compositions beneficially reduce the use of caustic beads. In such an embodiment, there may still be an initial step of concentrating an alkali metal hydroxide, such as by dissolving a solid alkali metal hydroxide in a liquid alkali metal hydroxide having 50% (actives basis) or less, to provide the concentrated alkali metal hydroxide.

The concentrated alkali metal hydroxide is present in an amount of about 1 wt.-% to about 99.9 wt.-%, about 10 wt.-% to about 90 wt.-%, about 20 wt.-% to about 90 wt.-%, about 30 wt.-% to about 90 wt.-%, about 40 wt.-% to about 90 wt.-%, about 50 wt.-% to about 90 wt .-%, about 60 wt.-% to about 90 wt.-%, or about 50 wt.-% to about 80 wt.-%. In other embodiments, the caustic solution includes from about 1 wt.-% to about 90 wt.-% of the total caustic to make the solid composition. In still other embodiments, the caustic solution includes from about 10 wt.-% to about 90 wt.-% of the total caustic to make the solid composition.

As described herein a liquid and a solid caustic can be combined to make the solid composition. In some embodiments where a solid caustic is employed the alkali metal hydroxide of the composition comprises less than about 40 wt-% solid caustic bead. In further embodiments where a solid caustic is employed, the composition has at least about 20%less solid caustic bead compared to a solid composition that does not contain the alkali metal hydroxide and reagent (e.g. propylene glycol).

### Polyol

The solid compositions contain at least one polyol to react with the alkali metal hydroxide to form the solid compositions. Polyols include C1-C22 alcohol, a glycol, or derivative thereof, or a combination thereof. In embodiments, a polyol is a diol, triol, and/or polyol containing more than 3 hydroxyl groups. Diols include for example, ethylene glycol, propylene glycol, hexylene glycol, tetramethylene glycol (1,4-Butanediol), etc. An exemplary triol is glycerin. An exemplary polyol is D-Sorbitol (6 hydroxyl groups).

Exemplary polyols include glycols and derivatives thereof including, ethylene glycol, propylene glycol, hexylene glycol, ethylene glycol phenyl ether, propylene glycol n-propyl ether, propylene glycol phenyl ether, dipropylene glycol n-propyl ether, and the like. Further exemplary glycerols and derivatives include, glycerol ethyl hexyl glyceryl ether, glycerin, glycerol formal, glycerol ketal, and the like. Exemplary polyols, diols and derivatives include, 3-butanediol, 1,4-butanediol, 2-ethy-1,3,-hexanediol, 1-3-propane diol, 2-methyl-2-propyl-1,3-propanediol, and the like.

A preferred polyol is glycerin. In an embodiment crude glycerin (˜85% active) is the preferred polyol. As the terms glycerin and glycerol may be used interchangeably.

According to various embodiments the polyol is included as a reagent to make the solid compositions in an amount of about 1 wt-% to about 30 wt-%, about 1 wt-% to about 20 wt-%, about 1 wt-% to about 15 wt-%, about 1 wt-% to about 10 wt-%, or about 2 wt-% to about 10 wt-%.

### Additional Functional Ingredients

The solid compositions may further include additional functional materials or additives that provide a beneficial property, e.g., for a particular use. Examples of conventional additives include one or more of each of salt or additional salt, chelant, alkalinity source, surfactant, detersive polymer, cleaning agent, rinse aid composition, softener, hydrotrope, pH modifier, source of acidity, anti-corrosion agent, secondary hardening agent, solubility modifier, detergent builder, detergent filler, defoamer, anti-redeposition agent, antimicrobial, rinse aids, a threshold agent or system, coagulant (e.g. sodium aluminate), aesthetic enhancing agent (i.e., dye, odorant, perfume), optical brightener, lubricant composition, bleaching agent, enzyme, effervescent agent, activator for an active oxygen compound, other such additives or functional ingredients, and the like, and mixtures thereof. Adjuvants and other additive ingredients will vary according to the type of composition being manufactured, and the intended end use of the solid composition.

According to various embodiments the additional functional ingredient(s) can be included as a reagent to make the solid compositions or included in the solid composition in an amount of about 0 wt-% to about 50 wt-%, about 0.1 wt-% to about 40 wt-%, about 0.5 wt-% to about 20 wt-%, about 1 wt-% to about 20 wt-%, or about 1 wt-% to about 10 wt-%.

In some embodiments, the compositions further include additional functional ingredients including one or more water conditioning agents (e.g. phosphinic acid polymers and an aminocarboxylate), nonionic surfactants (e.g. defoaming surfactant and/or wetting surfactant), coagulants, processing aids and/or chelant.

In some embodiments, the compositions further include a chelant (also referred to as a chelating agent). Examples of chelating agents include phosphonic acid and phosphonates, phosphates, aminocarboxylates and their derivatives, pyrophosphates, ethylenediamine and ethylenetriamine derivatives, hydroxyacids, and mono-, di-, and tri-carboxylates and their corresponding acids. In certain embodiments the composition is phosphate free. Preferred chelating agents include methylglycine-N,N-diacetic acid (MGDA); glutamic acid-N,N-diacetic acid (GLDA); ethylenediaminetetraacetic acid (EDTA); diethylenetriaminepentacetic acid (DTPA); nitrilotriacetic acid (NTA); Triethylenetetramine-N,N,N′,N″,N′″,N′″-hexaacetic acid (TTHA); Aspartic acid-N,N-diacetic acid (ASDA) and alkali, alkali earth metal, transition metal and/or ammonium salts thereof. In a further embodiment a biodegradable chelating agent, such as an aminocarboxylate is preferred.

In some embodiments, the compositions include a chelant comprising an aminocarboxylate selected from the group consisting of MGDA, NTA, EDTA, DTPA, and TTHA. In other embodiments, the compositions include a chelant comprising sodium gluconate. Without being limited to a particular mechanism of action the inclusion of a chelant, namely a biodegradable aminocarboxylate, benefits by increasing the melting point of the solid formed by a caustic/polyol/chelant by further bonding with the chelant.

In some embodiments, the compositions further include a detersive surfactant. Exemplary surfactants include nonionic surfactants including for example, EO/PO copolymers, reverse EO/PO copolymers, alcohol alkoxylates, including alcohol ethoxylates, pyrrolidones. Additional nonionic, anionic, amphoteric and zwitterionic surfactants are disclosed, for example in U.S. patent application Ser. No. 18/605,976, claiming priority to U.S. Ser. No. 63/490,857, filed simultaneously herewith and titled “Capped Block Copolymers, Their Synthesis, Manufacture, and Methods of Use”, which is incorporated by reference in its entirety.

In further embodiments, the compositions include a detersive surfactant or surfactant combination comprising a first surfactant comprising a reverse EO/PO block copolymer of about 20-40% EO for protein soil defoaming, and a second surfactant that is a reverse EO/PO block copolymer of about 40% EO, an alkyl capped alcohol ethoxylate (preferably a butyl capped alcohol ethoxylate), or alkyl pyrrolidone (preferably a C8, or C10 alkyl pyrrolidone) for protein soil removal from wares.

In some embodiments, the compositions further include a hydrotrope, viscosity modifier, solvent, water carrier, or derivatives or combinations thereof.

In some embodiments, the compositions further includes a water conditioning agent. The term “water conditioning agent” refers to a compound that inhibits crystallization of water hardness ions from solution or disperses mineral scale including but not limited to calcium carbonate. Water conditioning agents can include polymeric and small molecule water conditioning agents. Organic small molecule water conditioning agents are typically organocarboxylate compounds or organophosphate water conditioning agents. Polymeric water conditioning agents commonly comprise polyanionic compositions such as polyacrylic acid compounds.

Additional examples of water conditioning polymers includes polyacrylic acid homopolymer or alkali metal salt thereof, i.e., sodium polyacrylate. The polyacrylic acid homopolymers can contains a polymerization unit derived from the monomer selected from the group consisting of acrylic acid, methacrylic acid, methyl acrylate, methyl methacrylate, ethyl acrylate, ethyl methacrylate, butyl acrylate, butyl methacrylate, iso-butyl acrylate, iso-butyl methacrylate, iso-octyl acrylate, iso-octyl methacrylate, cyclohexyl acrylate, cyclohexyl methacrylate, glycidyl acrylate, glycidyl methacrylate, hydroxyethyl acrylate, hydroxypropyl acrylate, 2-hydroxyethyl acrylate, 2-hydroxyethyl methacrylate, 2-hydroxypropyl acrylate, 2-hydroxypropyl methacrylate. and hydroxypropyl methacrylate and a mixture thereof, among which acrylic acid. methacrylic acid, methyl acrylate, methyl methacrylate, butyl acrylate, butyl methacrylate, iso-butyl acrylate, iso-butyl methacrylate, hydroxyethyl acrylate, 2-hydroxyethyl acrylate, 2-hydroxyethyl methacrylate, 2-hydroxypropyl acrylate, and 2-hydroxypropyl methacrylate, and a mixture thereof are preferred.

Preferred are polyacrylic acids, (C₃H₄O₂)ₙ or 2-Propenoic acid homopolymers; Acrylic acid polymer; Poly(acrylic acid); Propenoic acid polymer; PAA have the following structural formula:

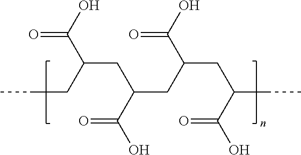

where n is any integer. One source of commercially available polyacrylates (polyacrylic acid homopolymers) includes the Acusol® 445 series from The Dow Chemical Company, Wilmington Delaware, USA, including, for example, Acusol® 445 (acrylic acid polymer, 48% total solids) (4500 MW), Acusol® 445N (sodium acrylate homopolymer, 45% total solids)(4500MW), and Acusol® 445ND (powdered sodium acrylate homopolymer, 93% total solids)(4500MW) Other polyacrylates (polyacrylic acid homopolymers) commercially available from Dow Chemical Company include, but are not limited to Acusol 929 (10,000 MW) and Acumer® 1510. Yet another example of a commercially available polyacrylic acid is AQUATREAT® AR-6 (100,000 MW) from AkzoNobel Strawinskylaan 2555 1077 ZZ Amsterdam Postbus 75730 1070 AS Amsterdam. Other suitable polyacrylates (polyacrylic acid homopolymers) include, but are not limited to those obtained from additional suppliers such as Aldrich Chemicals, Milwaukee, Wis., and ACROS Organics and Fine Chemicals, Pittsburg, Pa, BASF Corporation and SNF Inc. The homopolymers, copolymers, and/or terpolymers may be present in a composition from about 0.01 wt-% to about 30 wt-%. Maleic anhydride/olefin copolymers are copolymers of polymaleic anhydrides and olefins. Maleic anhydride (C₂H₂(CO)₂O has the following structure:

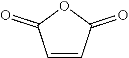

A part of the maleic anhydride can be replaced by maleimide, N-alkyl(C₁₋₄) maleimides, N-phenyl-maleimide, fumaric acid, itaconic acid, citraconic acid, aconitic acid, crotonic acid, cinnamic 10 acid, alkyl (C₁₋₁₈) esters of the foregoing acids, cycloalkyl(C₃₋₈) esters of the foregoing acids, sulfated castor oil, or the like. At least 95 wt % of the maleic anhydride polymers, copolymers, or terpolymers have a number average molecular weight of in the range between about 700 and about 20,000, preferably between about 1000 and about 100,000. A variety of linear and branched chain alpha-olefins can be used for the purposes of this disclosure. Particularly useful alpha-olefins are dienes containing 4 to 18 carbon atoms, such as butadiene, chloroprene, isoprene, and 2-methyl-1,5-hexadiene; 1-alkenes containing 4 to 8 carbon atoms, preferably C₄₋₁₀, such as isobutylene, 1-butene, 1-hexene, 1-octene, and the like.

In a preferred embodiment, particularly suitable maleic anhydride/olefin copolymers have a molecular weight between about 1000 and about 50,000, in a preferred embodiment between about 5000 and about 20,000, and in a most preferred embodiment between about 7500 and about 12,500. Examples of maleic anhydride/olefin copolymers which may be used include, but are not limited to, Acusol 460N from The Dow Chemical Company, Wilmington Delaware, USA. The maleic anhydride/olefin copolymer may be present in a composition from about 0.01 wt-% to about 30 wt-%.

Additional polymers include polycarboxylic acid polymers, include, but are not limited to, polymaleic acid homopolymers, polyacrylic acid copolymers, and maleic anhydride/olefin copolymers. Polymaleic acid (C₄H₂O₃)x or hydrolyzed polymaleic anhydride or cis-2-butenedioic acid homopolymer, has the structural formula:

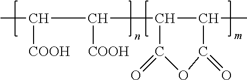

where n and m are any integer. Examples of polymaleic acid homopolymers, copolymers, and/or terpolymers (and salts thereof) which may be used are particularly preferred are those with a molecular weight of about 0 and about 5000, more preferably between about 200 and about 2000 (can you confirm these MWs). Commercially available polymaleic acid homopolymers include the Belclene 200 series of maleic acid homopolymers from BWATM Water Additives, 979 Lakeside Parkway, Suite 925 Tucker, GA 30084, USA and Aquatreat AR-801 available from AkzoNobel. The polymaleic acid homopolymers, copolymers, and/or terpolymers may be present in a composition from about 0.01 wt-% to about 30 wt-%.

Inorganic water conditioning agents include, but are not limited to, sodium tripolyphosphate and other higher linear and cyclic polyphosphates species. Suitable condensed phosphates include sodium and potassium orthophosphate, sodium and potassium pyrophosphate, sodium tripolyphosphate, and sodium hexametaphosphate. A condensed phosphate may also assist, to a limited extent, in solidification of the solid detergent composition by fixing the free water present in the composition as water of hydration. Examples of phosphonates included, but are not limited to: 1-hydroxyethane-1,1-diphosphonic acid, CH₃C(OH)[PO(OH)₂]₂; aminotri(methylenephosphonic acid), N[CH₂PO(OH)₂]₃; aminotri(methylenephosphonate), sodium salt (ATMP), N[CH₂PO(ONa)₂]₃; 2-hydroxyethyliminobis(methylenephosphonic acid), HOCH₂CH₂N[CH₂PO(OH)₂]₂; diethylenetriaminepenta(methylenephosphonic acid), (HO)₂POCH₂N[CH₂CH₂N[CH₂PO(OH)₂]₂]₂; diethylenetriaminepenta(methylenephosphonate), sodium salt (DTPMP), C₉H₂₈₋ₓN₃NaₓO₁₅P₅ (x=7); hexamethylenediamine(tetramethylenephosphonate), potassium salt, C₁₀H₂₈₋ₓN₂KₓO₁₂P₄(x=6); bis(hexamethylene)triamine(pentamethylenephosphonic acid), (HO₂)POCH₂N[(CH₂)₆N[CH₂PO(OH)₂]₂]₂; and phosphorus acid, H₃PO₃. A preferred phosphonate combination is ATMP and DTPMP. A neutralized or alkaline phosphonate, or a combination of the phosphonate with an alkali source before being added into the mixture such that there is little or no heat or gas generated by a neutralization reaction when the phosphonate is added is preferred.

In some embodiments, the compositions further include an anti-redeposition agent, namely that is capable of facilitating sustained suspension of soils in a cleaning or rinse solution and preventing removed soils from being redeposited onto the substrate being cleaned and/or rinsed. Some examples of suitable anti-redeposition agents can include fatty acid amides, fluorocarbon surfactants, complex phosphate esters, styrene maleic anhydride copolymers, and cellulosic derivatives such as hydroxyethyl cellulose, hydroxypropyl cellulose, and the like. A composition can include up to about 10 wt-%, and in some embodiments, in the range of about 1 to about 5 wt-%, of an anti-redeposition agent.

In some embodiments, the compositions further include a functional anhydrous material to absorb excess water from the mixture of hydrated solids in the composition. Examples of such a functional anhydrous material, include, but are not limited to, sodium carbonate (ash), sodium sulfate, solid caustic, and the like. Without being limited to a particular mechanism, the addition of a functional anhydrous material forms hydrate compounds upon contact with excess water, thus removing the excess water from the mixture.

METHODS OF MAKING SOLID COMPOSITIONS

The methods of making a solid composition include combining a concentrated alkali metal hydroxide and a polyol to generate in situ a solid composition, wherein the concentrated alkali metal hydroxide comprises greater than 50% (actives basis) liquid alkali metal hydroxide, and wherein the solid has a weight ratio of alkali metal hydroxide to water from about 60:40 to less than about 70:30, wherein the water is from any component of the solid composition and/or water added as a raw material, and wherein the solid is a cast solid, tablet, block, or powder, and wherein the solid is a dimensionally stable solid or a flowable powder. In preferred embodiments, the solid is a dimensionally stable solid.

The solid compositions are prepared by addition of materials. Without seeking to be limited to a particular theory of the disclosure, when the components of the composition are combined, the formation of high moles of solid hydrates occur as follows from reacting a polyol with a caustic source. In most embodiments, the methods of making the solid caustic compositions are equilibrium, non-stoichiometric reactions. The reagents for non-stoichiometric reactions include the alkali metal hydroxide and the polyol.

The formation of the solid compositions provides contiguous solid compositions, powders or granules comprising the solid caustic: water weight ratios disclosed herein. The solidification can be an in-situ generation. The methods of solidifying can include the step of combining the alkali metal hydroxide with the polyol, along with optionally combining at least one additional functional ingredient, and solidifying to form the solid composition. In preferred embodiments, the components reacted to form the solid composition are transferred into a container or housing before the solidification (e.g. capsule for dispensing). In some embodiments of the methods of making the solid compositions, the step of combining concentrated alkali metal hydroxide and polyol provides the components into a container or housing, such as a capsule or other container to form the solid cast, tablet, block, or powder, and preferably where the components are added to the capsule or container at a sufficiently high viscosity. If a composition viscosity is too low, phase separation can occur in the capsules. By increasing the viscosity, or even approaching a gel phase, the solution stays homogeneous and does not phase separate while cooling. In some embodiments, the desired viscosity at which to transfer the components into a container or housing is between about 500 cP to about 5000 cP, between about 1000 cP to about 4000 cP, or between about 800 cP to about 3000 cP to minimize or eliminate phase separation.

Beneficially, in embodiments the methods of solidifying do not require a step of chilling the composition quickly to lower the temperature below a solidification temperature, a significant benefit over commercial caustic bead-based solid compositions. In some embodiments no chilling step is included in the methods as complete solidification takes place without a chiller. In other embodiments, a chiller can optionally be utilized for faster processing. Beneficially, without a chilling step the solid compositions can lower below the solidification temperature at a faster rate than solid compositions having a caustic:water ratio of 69:31 or greater. This provides a further benefit of permitting transportation and holding more promptly.

In other embodiments, the reaction can form the solid compositions where solids can be isolated as a precipitate material for further use. Such further use may include a dehydration step to remove water. Such isolated and dehydrated solids are suitable for use in a solid composition or as a pre-mix. An exemplary application of use of a dehydrated solid binding composition would be for use in forming pressed solid compositions.

In some aspects, the compositions can be made by combining the components in an aqueous diluent using commonly available containers and blending apparatus. Beneficially, no special manufacturing equipment is required for making the compositions. A preferred method for manufacturing the solid compositions of the disclosure includes introducing the components into a stirred production vessel or a ribbon-mixing vessel.

In a method according to the disclosure, a composition is formed by mixing the reagents to form in situ a solid or to form a solid precipitate, and isolating the solid precipitates for further use. In such an embodiment, the solids precipitate can be further processed for use, such as for example spray drying the solid material, utilizing a fluid bed dryer, and/or through granulation. In other embodiments, the components can be processed in a ribbon blender to obtain a powdered form of the solid composition.

Spray drying is typically a one step process where a liquid is dried with hot gas and the resulting particles are often finer and do not have the opportunity to agglomerate or grow in size before reaching the product outlet.

In some embodiments where larger particles are desired, a fluid bed can be integrated into the process. In fluid bed drying, a liquid is dried with hot gas and the resulting particles have the opportunity to recirculate through the spray zone enabling agglomeration or layered growth of the particles to a desired size. Particles of these sizes are often referred to as granulates. Granulation is a broader term where through various methods or pieces of equipment, a finer powder is grown into granulates. Fluid bed granulation is a type of granulation.

In an embodiment, the dried material is a powder or granule form of the solid. This beneficially provides a dust-free, free-flowing granule or powder that has high alkoxide activity. In an exemplary embodiment, the alkoxide solidification reaction is completed in an aqueous solution and then dried (e.g. through spray drying, fluid bed dryer, or granulation) to further concentrate the caustic solids in powder or granulate form.

In a further method according to the disclosure, a composition is formed by mixing the reagents and forming a contiguous one phase homogenous solid.

In a still further method, the solid composition is formed by mixing reagents and subjecting the mixture to an additional energy input, such as to create a molten concentrated alkali metal hydroxide or such as generating a cast solid that takes place at elevated temperature to avoid premature solidification. The optional additional energy may be used in the form of heat, radiation, and the like. In a preferred embodiment, the additional energy is microwaving for a short period of time. In exemplary embodiments, microwave energy has frequencies above about 300 megahertz (hereinafter often abbreviated as “MHz”), and are generally regarded as having frequencies in the range of about 300 to about 300,000 MHz. Preferred methods of microwaving are disclosed in U.S. Pat. Nos. 5,858,299 and 6,689,305, each of which are herein incorporated by reference in its entirety.

In embodiments employing heat to the reaction can include concentrating the alkali metal hydroxide and/or adding heat (i.e. microwave) to remove water from the solid (caustic:water) to speed up/enhance solidification.

In an embodiment of the method, the additional energy input occurs for a time period of less than 5 minutes, preferably less than 2 minutes, preferably less than 1 minute, and preferably less than 45 seconds. The compositions of the present disclosure can be formed by combining the components in the weight percentages and ratios disclosed herein.

The solid compositions beneficially can reduce (or eliminate) the use of solid caustic inputs (i.e. caustic beads) in a solid composition. In an embodiment, at least about 40%, 45%, 50%, 55%, 60%, 65%, 70%, 75%, 80%, 85%, 90%, 95%, or 100% reduction of solid caustic is achieved. As described in certain embodiments a 100% reduction (i.e. elimination) of caustic beads is achieved.

Compositions can be produced using a batch or continuous mixing system. In an exemplary embodiment, a single-or twin-screw extruder is used to combine and mix one or more agents at high shear to form a homogeneous mixture. In some embodiments, the processing temperature is at or below the melting temperature of the components. In some embodiments, the processing temperature is at ambient temperature (20-25° C.). In embodiments employing a concentrated alkali metal hydroxide an elevated temperature is used for the processing temperature (66-85° C.). The processed mixture may be dispensed from the mixer by forming, casting or other suitable means, whereupon the composition hardens to a solid form. The structure of the matrix may be characterized according to its hardness, melting point, material distribution, crystal structure, and other like properties according to known methods in the art. Generally, a solid composition processed according to the methods is substantially homogeneous with regard to the distribution of ingredients throughout its mass and is dimensionally stable.

Specifically, in a forming process, the liquid and/or solid components are introduced into the mixing system and are continuously mixed until the components form a contiguous block. In an exemplary embodiment, the components are mixed in the mixing system for at least approximately 5 seconds, 15 seconds, 30 seconds, 60 seconds, 5 minutes, 10 minutes, 30 minutes, 60 minutes. The mixture is then discharged from the mixing system. In an embodiment, the mixture can be discharged into, or through, a die or other shaping means. The product is then packaged.

In an exemplary embodiment, the formed composition begins to harden to a solid form in between approximately 1 minute and approximately 3 hours. Particularly, the formed composition begins to harden to a solid form in between approximately 1 minute and approximately 2 hours. More particularly, the formed composition begins to harden to a solid form in between approximately 1 minute and approximately 20 minutes.

Pressing can employ low pressures compared to conventional pressures used to form tablets or other conventional solid compositions. For example, in an embodiment, the present method employs a pressure on the solid of only less than or equal to about 5000 psi. In certain embodiments, the present method employs pressures of less than or equal to about 3500 psi, less than or equal to about 2500 psi, less than or equal to about 2000 psi, or less than or equal to about 1000 psi. In certain embodiments, the present method can employ pressures of about 1 to about 1000 psi, about 2 to about 900 psi, about 5 psi to about 800 psi, or about 10 psi to about 700 psi.

Specifically, in a casting process, the liquid and solid components are introduced into the final mixing system and are continuously mixed until the components form a substantially homogeneous liquid mixture in which the components are distributed throughout its mass. In an exemplary embodiment, the components are mixed in the mixing system for at least approximately 60 seconds. Once the mixing is complete, the product is transferred to a packaging container where solidification takes place. In an exemplary embodiment, the cast composition begins to harden to a solid form in between approximately 1 minute and approximately 3 hours. Particularly, the cast composition begins to harden to a solid form in between approximately 1 minute and approximately 2 hours. More particularly, the cast composition begins to harden to a solid form in between approximately 1 minute and approximately 20 minutes.

By the term “solid form”, it is meant that the hardened composition will not flow and will substantially retain its shape under moderate stress or pressure or mere gravity. The degree of hardness of the solid cast composition may range from that of a fused solid product which is relatively dense and hard, for example, like concrete, to a consistency characterized as being a hardened paste. In addition, the term “solid” refers to the state of the composition under the expected conditions of storage and use of the solid composition. In general, it is expected that the composition will remain in solid form when exposed to temperatures of up to approximately 100° F and particularly greater than approximately 120° F.

The resulting solid composition may take forms including, but not limited to: a pressed solid; a cast solid product; an extruded, molded or formed solid pellet, block, tablet, powder, granule, flake; or the formed solid can thereafter be ground or formed into a powder, granule, or flake. In an exemplary embodiment, extruded pellet materials formed have a weight of between approximately 1 gram and 50 grams, or 50 grams and approximately 250 grams, extruded solids generally have a weight of approximately 100 grams or greater, and solid blocks generally have a mass of between approximately 1 and approximately 10 kilograms.

The solid compositions provide for a stabilized source of functional materials. In some embodiments, the solid composition may be dissolved, for example, in an aqueous or other medium, to create a concentrated and/or use solution. The solution may be directed to a storage reservoir for later use and/or dilution, or may be applied directly to a point of use. Alternatively, the solid alkaline composition is provided in the form of a unit dose, typically provided as a cast solid, an extruded pellet, or a tablet having a size of between approximately 1 gram and approximately 100 grams. In another alternative, multiple-use solids can be provided, such as a block or a plurality of pellets, and can be repeatedly used to generate aqueous compositions for multiple cycles.

### Methods of Use

The methods of using the solid compositions described herein include generating a use solution of the solid composition, contacting an article or surface in need of cleaning, disinfecting, and/or sanitizing with the use solution, and cleaning, disinfecting, and/or sanitizing the article or surface. In embodiments, the solid composition does not slough during dispensing to generate the use solution.

It is a benefit of the solid compositions comprising less than about 70:30 alkali metal hydroxide to water weight ratio provide improved cleaning performance in comparison to compositions comprising additional alkali metal hydroxide. The solid compositions are less concentrated caustic compositions and therefore allow for additional performance additives (e.g. chelants, surfactants) to improve the cleaning performance. For example, in an embodiment the solid compositions can contain less active caustic (after neutralization from acidic polymers) in the composition compared to inline solid caustic detergents and provide at least the same or improved cleaning efficacy.

The present disclosure includes methods of using the solid compositions for various cleaning applications. These cleaning compositions can operate on an article, surface, in a body or stream of water or a gas, or the like, by contacting the article, surface, body, or stream with a composition of the disclosure. Contacting can include any of numerous methods for applying a cleaning composition of the disclosure, such as spraying the compositions, immersing the article in compositions, foam or gel treating the article with the compounds or composition, or a combination thereof.

It should be understood that the concentration of the ingredients in the solid compositions will vary depending on whether the cleaning composition is provided as a concentrate or as a use solution. A use solution may be prepared from the concentrate by diluting the concentrate with water at a dilution ratio that provides a use solution having desired detersive properties.

In some embodiments, the cleaning compositions are used in methods for cleaning soiled surfaces via use of a degreaser composition. In one embodiment, the present disclosure is a method for cleaning polymerized fat soils. The cleaning methods generally use non-corrosive degreaser compositions incorporating the compositions as described herein. In certain embodiments, an environmental cleaning method is provided. In other embodiments, a clean in place (CIP) method is provided. According to further embodiments of the invention, non-corrosive degreaser compositions can be used in any other methods seeking to remove polymerized soils without requiring the use of corrosive formulations, such as removing polymerized or cross-linked films from floors and other finishes. Beneficially, degreaser compositions do not require use of personal protective equipment as a result of the pH below about 11.5. In addition, the degreaser compositions achieve degreasing action within approximately 5 seconds to a few minutes of contact to a soiled surface. According a preferred embodiment of the invention, application of degreaser compositions result in soil removal within about 10 seconds without requiring substantial mechanical action or excessive temperatures. The methods of the present disclosure result in cleaning efficacy at least the same as that obtained with the use of corrosive, highly alkaline compositions of the prior art.

Exemplary industries in which the present methods can be used include, but are not limited to: food service industry; food and beverage industry; consumer degreasing applications; oil processing industry; industrial agriculture and ethanol processing; and the pharmaceutical manufacturing industry. Suitable uses for the compositions and methods of the invention may include, for example, oven cleaner, including microwave ovens, general degreaser, fryer degreaser, smokehouse cleaner, floor cleaner, exhaust hood cleaner, drain cleaner, floor finish remover, floor cleaner, fryer cleaner, pot and pan cleaner, carpet spotter, pharmaceutical and cosmetics cleaner, instrument cleaner, tar remover, and the like.

The present methods can also be used to remove soils other than polymerized soils, such as those removed with degreaser compositions. Such other soils include, but are not limited to, starch, cellulosic fiber, protein, simple carbohydrates and combinations of any of these soil types with mineral complexes. Examples of specific food soils that are effectively removed using the present methods include, but are not limited to, soils generated in the manufacture and processing meat, poultry, vegetables and fruit, bakery goods, soft drinks, brewing and fermentation residues, soils generated in sugar beet and cane processing and processed foods containing these ingredients and associated ingredients such as juices, sauces and condiments. These soils can develop on environmental surfaces such as walls and floors, freezers and cooling systems, heat exchange equipment surfaces, conveyor surfaces and on other surfaces during the manufacturing and packaging process.

In further embodiments, the methods of employing cleaning compositions are particularly suited for use in closed systems, e.g. dish or ware washing systems for cleaning, sanitizing and/or disinfecting articles and surfaces.

The method includes contacting an article or surface with a cleaning composition or a cleaning use composition to wash the surface. The method can contact the liquid to any of a variety of surfaces or objects including surfaces or articles including those made of glass, ceramic, plastic, porcelain, aluminum, or the like.

The phrase “washing a surface with a wash solution (or a use solution or a cleaning composition)” refers to the circulation of a cleaning composition solution to remove substantially all soil from the treated surfaces (e.g. ware) and to keep that soil suspended or dissolved. In an embodiment, this step may be conducted where the temperature of the rinse water is up to about 140° F., preferably in the range of 100° F. to 140° F., preferably in the range of 110° F. to 140° F., and most preferably in the range of 120° F. to 140° F. As referred to herein, “low temperature” refers to those rinse water temperatures below about 140° F. For example, conventional rinse temperature for ware washing occurs above 140° F., such as from about 140° F. to about 190° F., particularly between about 145° F. to about 180° F. In an aspect, the methods employing a low temperature further employ a sanitizer.

Contacting can include any of numerous methods for applying a cleaning composition, such as spraying the composition, immersing the object in the composition, or a combination thereof. A concentrate or use concentration of a composition can be applied to or brought into contact with an article by any conventional method or apparatus for applying a cleaning composition to an object. For example, the object can be wiped with, sprayed with, and/or immersed in the composition, or a use solution made from the composition. The composition can be sprayed, or wiped onto a surface; the composition can be caused to flow over the surface, or the surface can be dipped into the composition. Contacting can be manual or by machine.

Before contacting an article or surface, a concentrate cleaning composition may be first diluted with water at the location of use to provide the use solution. When the composition is used in an automatic warewashing or dishwashing machine, it is expected that that the location of use will be inside the automatic warewashing machine. Depending on the machine, the composition may be provided in a unit dose form or in a multi-use form. In larger warewashing machines, a large quantity of composition may be provided in a compartment that allows for the release of a single dose amount of the composition for each wash cycle. Such a compartment may be provided as part of the warewashing machine or as a separate structure connected to the warewashing machine.

The cleaning composition may also be dispensed from a spray-type dispenser, such as that disclosed in U.S. Pat. Nos. 4,826,661, 4,690,305, 4,687,121, 4,426,362 and in U.S. Pat. Nos. Reissue 32,763 and 32,818, the disclosures of which are incorporated by reference herein. Briefly, a spray-type dispenser functions by impinging a water spray upon an exposed surface of the composition, and then immediately directing the use solution out of the dispenser to a storage reservoir or directly to a point of use. If necessary, in some embodiments, when used, the product may be removed from the packaging and inserted into the dispenser. The methods may further employ one or more rinse steps for the treated articles or

surfaces. In an aspect, the commercial use of the phosphorus-free detergent compositions at low temperatures preferably include a rinse step employing a rinse aid, including for example, the disclosure of using rinse aids set forth in U.S. patent application Ser. No. 13/480,031, which is herein incorporated by reference in its entirety.

In some embodiments, the cleaning compositions include killing one or more of the pathogenic bacteria associated with health care surfaces and environments including, but not limited to, Salmonella typhimurium, Staphylococcus aureus, methicillin resistant Staphylococcus aureus, Salmonella choleraesurus, Pseudomonas aeruginosa, Escherichia coli, mycobacteria, yeast, and mold. The cleaning compositions have activity against a wide variety of microorganisms such as Gram positive (for example, Listeria monocytogenes or Staphylococcus aureus) and Gram negative (for example, Escherichia coli or Pseudomonas aeruginosa) bacteria, yeast, molds, bacterial spores, viruses, etc. The compounds and compositions of the present disclosure, as described above, have activity against a wide variety of human pathogens. The cleaning compositions can kill a wide variety of microorganisms on a food processing surface, on the surface of a food product, in water used for washing or processing of food product, on a health care surface, or in a health care environment.

The present methods can be used to achieve any suitable reduction of the microbial population in and/or on the target or the treated target composition. In some embodiments, the present methods can be used to reduce the microbial population in and/or on the target or the treated target composition by at least one log10. In other embodiments, the present methods can be used to reduce the microbial population in and/or on the target or the treated target composition by at least two log10. In still other embodiments, the present methods can be used to reduce the microbial population in and/or on the target or the treated target composition by at least three log10. In still other embodiments, the present methods can be used to reduce the microbial population in and/or on the target or the treated target composition by at least five log10. Without limiting the scope of disclosure, the numeric ranges are inclusive of the numbers defining the range and include each integer within the defined range.

The cleaning compositions can be used for a variety of domestic or industrial applications, e.g., to reduce microbial or viral populations on a surface or object or in a body or stream of water. The cleaning compositions can be applied in a variety of areas including kitchens, bathrooms, factories, hospitals, dental offices and food plants, and can be applied to a variety of hard or soft surfaces having smooth, irregular or porous topography. Suitable hard surfaces include, for example, architectural surfaces (e.g., floors, walls, windows, sinks, tables, counters and signs); eating utensils; hard-surface medical or surgical instruments and devices; and hard-surface packaging. Such hard surfaces can be made from a variety of materials including, for example, ceramic, metal, glass, wood or hard plastic. Suitable soft surfaces include, for example paper; filter media; hospital and surgical linens and garments; soft-surface medical or surgical instruments and devices; and soft-surface packaging. Such soft surfaces can be made from a variety of materials including, for example, paper, fiber, woven or nonwoven fabric, soft plastics and elastomers. The cleaning compositions can also be applied to soft surfaces such as food and skin (e.g., a hand). The present compounds can be employed as a foaming or non-foaming environmental sanitizer or disinfectant.

The cleaning compositions can be included in products such as degreasers, sterilants, sanitizers, disinfectants, preservatives, deodorizers, antiseptics, fungicides, germicides, sporicides, virucides, detergents, bleaches, hard surface cleaners, hand soaps, waterless hand sanitizers, lubricants, rinse aids, 2-in-1 and/or 3-in-1 products, such as insecticide/cleaner/sanitizer, 3-sink applications, and pre-or post-surgical scrubs.

The cleaning compositions can be applied to microbes or to soiled or cleaned surfaces using a variety of methods. These methods can operate on an object, surface, in a body or stream of water or a gas, or the like, by contacting the object, surface, body, or stream with a compound of the disclosure. Contacting can include any of numerous methods for applying a compound, such as spraying the compound, immersing the object in the compound, foam or gel treating the object with the compound, or a combination thereof.

A concentrate or use concentration of a cleaning composition can be applied to or brought into contact with an object by any conventional method or apparatus for applying an antimicrobial or cleaning compound to an object. For example, the object can be wiped with, sprayed with, foamed on, and/or immersed in the compound, or a use solution made from the composition. The cleaning composition can be sprayed, foamed, or wiped onto a surface; the composition can be caused to flow over the surface, or the surface can be dipped into the cleaning composition. Contacting can be manual or by machine. Food processing surfaces, food products, food processing or transport waters, and the like can be treated with liquid, foam, gel, aerosol, gas, wax, solid, or powdered stabilized compounds according to the disclosure, or solutions containing these compounds.

Cleaning compositions of the disclosure can be formulated and sold for use as is, or as solvent or solid concentrates. If desired, such concentrates can be used full-strength as sanitizing rinse compositions. However, the concentrates typically will be diluted with a fluid (e.g., water) that subsequently forms the dilute phase or a use solution. Preferably, the concentrate forms a single phase before such dilution and remains so while stored in the container in which it will be sold. When combined with water or other desired diluting fluid at an appropriate dilution level and subjected to mild agitation (e.g., by stirring or pumping the composition), some compositions of the disclosure will form a pseudo-stable dispersion, and other compositions of the disclosure will form a clear or quasi-stable solution or dispersion. If a pseudo-stable composition is formed, then the composition preferably remains in the pseudo-stable state for a sufficiently long period so that the composition can be applied to a surface before the onset of phase separation. The pseudo-stable state need only last for a few seconds when suitably rapid application techniques such as spraying are employed, or when agitation during application is employed. The pseudo-stable state desirably lasts for at least one minute or more after mixing and while the composition is stored in a suitable vessel, and preferably lasts for five minutes or more after mixing. Often normal refilling or replenishment of the applicator (e.g., by dipping the applicator in the composition) will provide sufficient agitation to preserve the pseudo-stable state of the composition during application.

The various applications of use described herein provide the cleaning compositions to a surface and/or water source. Beneficially, the cleaning compositions of the disclosure are fast-acting. However, the present methods require a certain minimal contact time of the compositions with the surface or product in need of treatment for occurrence of sufficient antimicrobial effect. The contact time can vary with concentration of the use compositions, method of applying the use compositions, temperature of the use compositions, pH of the use compositions, amount of the surface or product to be treated, amount of soil or substrates on/in the surface or product to be treated, or the like. The contact or exposure time can be about 15 seconds, at least about 15 seconds, about 30 seconds or greater than 30 seconds. In some embodiments, the exposure time is about 1 to 5 minutes. In other embodiments, the exposure time is a few minutes to hours. In other embodiments, the exposure time is a few hours to days. The contact time will further vary based upon the use concentration of actives of compositions according to the disclosure.

### Embodiments

The present disclosure is further defined by the following numbered paragraphs:

[Paragraph 1] A solid composition comprising: an alkali metal hydroxide; a polyol; and water; wherein the solid has a weight ratio of alkali metal hydroxide to water from about 60:40 to less than about 70:30, wherein the water is from any component of the solid composition and/or water added as a raw material; and wherein the solid is a contiguous solid, powder or granule. The solid composition of paragraph 1, wherein the alkali metal hydroxide is sodium hydroxide, potassium hydroxide, lithium hydroxide, derivatives thereof, or combinations thereof.

The solid composition of any one of paragraphs 1-2, wherein the polyol is a C1-C22 alcohol, a glycol, or derivative thereof, or a combination thereof.

The solid composition of paragraphs 1-2, wherein the polyol is a diol, triol, and/or polyol containing more than 3 hydroxyl groups.

The solid composition of any one of paragraphs 1-4, wherein the polyol is a glycol and/or glycerin.

The solid composition of paragraph 5, wherein the glycol is an aliphatic glycol comprising ethylene glycol, propylene glycol, hexylene glycol, 1,4-butanediol, or combinations thereof.

The solid composition of any one of paragraphs 1-6, further comprising detersive surfactant, water conditioning agent, chelating agent, hydrotrope, carbonate alkalinity source, anhydrous material to absorb excess water in the solid composition, or combinations thereof.

The solid composition of paragraph 7, wherein: (i) the detersive surfactant or surfactant combination comprises a first surfactant comprising a reverse EO/PO block copolymer of about 20-40% EO for protein soil defoaming, and a second surfactant that is a reverse EO/PO block copolymer of about 40% EO, an alkyl capped alcohol ethoxylate (preferably a butyl capped alcohol ethoxylate), or alkyl pyrrolidone (preferably a C8, or C10 alkyl pyrrolidone) for protein soil removal from wares; (ii) the chelating agent is an aminocarboxylate (preferably MGDA); (iii) the chelating agent is an aminocarboxylate selected from the group consisting of MGDA, NTA, EDTA, DTPA, and TTHA; and/or (iii) the chelating agent is Sodium Gluconate.

The solid composition of any one of paragraphs 1-8, wherein the solid is a cast solid, and wherein the solid is dimensionally stable.

[Paragraph 10] A method of making a solid composition comprising: combining a concentrated alkali metal hydroxide and a polyol to generate in situ a solid composition, wherein the concentrated alkali metal hydroxide comprises greater than 50% (actives basis) liquid alkali metal hydroxide, and wherein the solid has a weight ratio of alkali metal hydroxide to water from about 60:40 to less than about 70:30, wherein the water is from any component of the solid composition and/or water added as a raw material, and wherein the solid is a cast solid, tablet, or block, and wherein the solid is a dimensionally stable solid.

The method of paragraph 10, wherein the method further comprises an initial step of concentrating an alkali metal hydroxide by either (i) concentrating a liquid alkali metal hydroxide having 50% (actives basis) or less alkali metal hydroxide by evaporation, or (ii) by dissolving a solid alkali metal hydroxide in a liquid alkali metal hydroxide having 50% (actives basis) or less, to provide the concentrated alkali metal hydroxide.

The method of any one of paragraphs 10-11, wherein the step of combining concentrated alkali metal hydroxide and polyol provides the components into a capsule or container to form the solid cast, tablet, block, or powder, and preferably where the components are added to the capsule or container at a sufficiently high viscosity.

The method of any one of paragraphs 10-12, wherein the concentrated alkali metal hydroxide is from about 69% to about 74% (actives basis) liquid alkali metal hydroxide, preferably from about 70% to about 73% (actives basis) liquid alkali metal hydroxide.

The method of any one of paragraphs 10-13, wherein the method does not require the use of a chiller for solidification, or does not use a chiller for solidification.

The method of any one of paragraphs 10-14, wherein the solid composition is formed between about 1 minute and about 3 hours, or between about 1 minute and about 20 minutes.

[Paragraph 16] A method of using a solid composition comprising: generating a use solution of the solid composition according to any one of paragraphs 1-9 or the composition made by the methods of any one of claims 10-15; contacting an article or surface in need of cleaning, disinfecting, and/or sanitizing with the use solution; and cleaning, disinfecting, and/or sanitizing the article or surface.

The method of paragraph 16, wherein the solid composition does not slough during dispensing to generate the use solution.

The method of any one of paragraphs 16-17, wherein the solid composition comprises less than about 70:30 alkali metal hydroxide to water ratio and provides improved cleaning performance in comparison to compositions comprising additional alkali metal hydroxide.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

The following Examples provide exemplary embodiments of the solid compositions formed according to the methods described herein.

The following materials were employed in the Examples:
PSO: Phosphinosuccinic Oligomer
Nalco Exp 929: 85% Acrylic Acid, 10% Maleic Acid, 5% ATBS) water conditioning polymer
90R4 or Tetronic 90R4: tetrafunctional block copolymer with terminal secondary hydroxyl group
Genapol BE 2410: lauryl fatty alcohol ethoxylate butylether
Acusol 445ND: homopolymer of acrylic acid, fully neutralized, spray dried detergent polymer
Acusol 448: 3000 MW polyacrylic:polymaleic copolymer
Pluronic 25R2: long chain EO/PO block copolymer
Surfadone LP-100: vinyl ether polymers and copolymers
SURFONIC LD-097: Polyoxypropylene Polyoxyethylene Block Copolymer
Acusol 810A: Alkali Soluble acrylic polymer Emulsion (ASE)

### Example 1

The use of higher active caustic liquid for the control of equilibrium/kinetics of solidification was evaluated. Fractions of propylene glycol were slowly and sequentially added to a fixed volume of 50% NaOH with mixing to obtain to a 1:1 molar ratio. The observations are described in Table 2.

TABLE 2

| Molar ratio of NaOH to | |
|---|---|
| propylene glycol | Observation |
| Around 10:1 to 5:1 | Rapid formation of “large slabs/chunks” |
| | of solids. |
| | Exotherm reaction. |
| | These chunks of solids can be broken to |
| | smaller pieces by mixing. |
| Middle stages | The solids start to re-dissolve. |
| 1:1 final point | Viscous liquid. |
| | Upon sitting for a long duration, such as |
| | overnight, the composition gradually |
| | solidifies. |

Without being limited to a particular mechanism of action, the use of higher active caustic liquid for the solidification of the caustic results in a more rapid and complete solidification according to Le Chatelier's principle. As the sodium hydroxide is used up in the equilibrium, the reaction slows down and does not go to completion. At some point, there is not enough caustic activity to insolubilize the propylene glycol to facilitate the solidification-here, propylene glycoxide. The propylene glycol becomes more soluble and can re-dissolve the formed propylene glycoxide (see Table at middle stages). At the final point of about 1:1 molar ratio, as the remaining propylene glycol in reaction is allowed to sit for a long time (e.g. overnight) the viscous liquid solidifies slowly, caustic activity goes up and reaction is driven towards completion.

Using Le Chatelier's principal, the reaction can be driven towards completion by using higher active caustic liquid, such as 70% NaOH (which is a liquid ≥149° F and within cast solid manufacturing conditions).

### Example 2

Various solid compositions including an alkali metal hydroxide, a polyol, water, and additional functional ingredients were made to assess solidification of compositions with a weight ratio of alkali metal hydroxide to water ranging between about 66:34 to about 67.5:32.5 using 72% molten sodium hydroxide made by combining caustic beads with water. For example, 77.78 wt-% of 72% molten caustic solution can be made by combining 56% caustic beads with 21.8% water. The studies evaluated the in-situ solidification of the compositions using 72% molten sodium hydroxide to assess hardness and other solidification attributes. Test compositions were made in the laboratory as summarized in Table 3 and the solidification observations are summarized in Table 4.

TABLE 3

| | Test Composition | | | | | |
|---|---|---|---|---|---|---|
| Component | 1 | 2 | 3 | 4 | 5 | 6 |
| 72% NaOH | 77.94 | 77.94 | 77.77 | 80.55 | 80.55 | 80.55 |
| Solution | | | | | | |
| Crude | 6 | 6 | 6.17 | 6.17 | 6.17 | 6.17 |
| glycerin (85%) | | | | | | |
| Hexylene glycol | 0.1 | 0.1 | 0.1 | 0.1 | 0.1 | 0.1 |
| PSO | 1.65 | 1.65 | 1.65 | 1.72 | 5.72 | 5.72 |
| Nalco Exp 929 | 7.6 | 7.6 | 7.6 | 8 | | |
| 25RS | 1 | 1 | 0.75 | 0.75 | 0.75 | 0.75 |
| 90R4 | 1.5 | 1.5 | | | | |
| Genapol BE | | | 2.5 | 2.5 | 2.5 | |
| 2410 | | | | | | |
| Acusol 445ND | | | | | 4 | 4 |
| powder | | | | | | |
| Sodium Aluminate | 0.21 | 0.21 | 0.21 | 0.21 | 0.21 | 0.21 |
| Dense ash | 4 | 4 | 3.25 | | | 2.5 |
| (sodium carbonate) | | | | | | |
| Caustic:water | 66.3:33.7 | 66.3:33.7 | 66.0:34.0 | 66.0:34.0 | 67.3:32.7 | 67.5:32.5 |
| wt-ratio | | | | | | |
| Total | 100 | 100 | 100 | 100 | 100 | 100 |

TABLE 4

| | Test Compositions | | | | | |
|---|---|---|---|---|---|---|
| | 1 | 2 | 3 | 4 | 5 | 6 |
| Observations | Gel state | Gel state | Collected | Small gel-like | Some foam | Ash settled at |
| | added to | added to | wet ash at | particles at | aggregation | bottom. |
| | container | container for | the bottom | bottom; | (may be due | The solid |
| | for cast | cast solid; | of container; | poured | to crude | has no phase |
| | solid | sodium | confirming | together | glycerin at | separation. |
| | | carbonate at | solidification | with | the last step) | |
| | | bottom of | does not | mixture into | during mixing. | |
| | | container; | require the | container | Phase separation | |
| | | variations in | sodium | for case | on top layer | |
| | | mix order | carbonate. | solid | (soft) with | |
| | | did not change | | | bottom layer | |
| | | solidification | | | hard solid. | |

Test composition 1 made a 300 gram test formulation that was suitable for adding to a container or mold for cast solidification.

Test compositions 2-6 were used to make 1000 gram test formulations using overhead mixing. As summarized in the observations the 72% molten caustic is used to produce an in-situ cast solid, where sodium carbonate (ash) is not required for solidification and mix order of the components is not essential to solidification.

### Example 3

Additional testing was completed assessing solidification using the 72% caustic solution made by a lower caustic level of 48% beads. As summarized in the Table 4, some formulations used dried forms of PSO and Acusol 448, respectively labeled “PSO Powder” and “Acusol 448 Powder” which are not the same as Acusol 445 ND (dried polymer that is pre-neutralized).

Variations in the polymers were evaluated to assess impact of the decreased water content of the powdered forms on the formation of the solid compositions. A 300 gram trial was completed in the laboratory as summarized in Table 5 and the solidification observations are summarized in Tables 6-7.

The summarized observations show the trend towards incomplete solidification or soft solids when the caustic: water ratio in the solid is far below the 60:40. Those compositions near the caustic: water ratio of 60:40 were still complete solids although softer than those having a higher caustic:water ratio were harder solids

TABLE 5

| | Test Composition | | | | | | | |
|---|---|---|---|---|---|---|---|---|
| Component | 7 | 8 | 9 | 10 | 11 | 12 | 13 | 14 |
| 72% naOH | 66.67 | | 80.23 | 75 | 73 | 73 | 72.93 | 80.6 |
| Solution | | | | | | | | |
| 50% NaOH | | 55.73 | | | 2 | 2 | 2 | |
| Crude glycerin | 12 | 9.8 | 6.17 | 6 | 6 | 6 | 5.99 | 6 |
| (85%) | | | | | | | | |
| Hexlene glycol | 0.1 | 0.1 | 0.1 | 0.1 | 0.1 | 0.1 | 0.1 | 0.1 |
| PSO | 5.75 | | 5.74 | 6.8 | 6.8 | 6.8 | 6.79 | |
| PSO Powder | | 7.84 | | | | | | 4.39 |
| Acusol 448 | 6 | | 6.8 | 5.74 | 5.74 | 5.74 | 5.73 | |
| Acusol 448 | | | | | | | | 5.2 |
| Powder | | | | | | | | |
| 25R2 | 0.75 | 0.73 | 0.75 | 0.75 | 0.75 | 0.75 | 0.75 | 1 |
| Genapol BE | 2.5 | 2.45 | | 2.4 | 2.4 | 2.4 | | |
| 2410 | | | | | | | | |
| Surfadone L100 | | | | | | | | |
| LO097 | | | | | | | 2.5 | 2.5 |
| Acusol 445RD | | 6.7 | | | | | | |
| powder | | | | | | | | |
| Sodium | 0.21 | 0.21 | 0.21 | 0.21 | 0.21 | 0.21 | 0.21 | 0.21 |
| Aluminate | | | | | | | | |
| Dense ash | 5.02 | | | | 3 | | 3 | |
| (sodium | | | | | | | | |
| carbonate) | | | | | | | | |
| Light Ash | | | | 3 | | | | |
| (sodium | | | | | | | | |
| carbonate) | | | | | | | | |
| Sodium | | 10.88 | | | | | | |
| Gluconate | | | | | | | | |
| Sulfate | | 5.47 | | | | | | |
| Acusol 810A | | | | | | 3 | | |
| Propylene | | | | | | | | |
| Glycol | | | | | | | | |
| Sodium Citrate | | | | | | | | |
| MGDA | | | | | | | | |
| Caustic:water | 61.8:38.2 | 48.0:52.0 | 64.2:35.8 | 63.6:36.4 | 63.1:36.4 | 60.6:39.4 | 63.3:36.7 | 68.8:31.2 |
| wt-ratio | | | | | | | | |
| Total | 100 | 100 | 100 | 100 | 100 | 100 | 100 | 100 |

| | Test Composition | | | | | | |
|---|---|---|---|---|---|---|---|
| Component | 15 | 16 | 17 | 18 | 19 | 20 | 21 |
| 72% naOH | 72.93 | 74.93 | 75.17 | 75.71 | | 74.93 | 74.93 |
| Solution | | | | | | | |
| 50% NaOH | 2 | | | | 64.1 | | |
| Crude glycerin | 5.99 | 5.99 | 6.01 | 5.99 | | 5.99 | 5.99 |
| (85%) | | | | | | | |
| Hexlene glycol | 0.1 | 0.1 | 0.1 | 0.1 | 0.1 | 0.1 | 0.1 |
| PSO | 5.73 | 5.73 | 9.81 | | | 5.73 | 5.73 |
| PSO Powder | | | | 4.4 | 6.75 | | |
| Acusol 448 | 6.79 | 6.79 | | 10.09 | | 6.79 | 6.79 |
| Acusol 448 | | | 5.2 | | | | |
| Powder | | | | | | | |
| 25R2 | 0.75 | 0.75 | 1 | 1 | 0.75 | 0.75 | 0.75 |
| Genapol BE | | | | 2.5 | | | |
| 2410 | | | | | | | |
| Surfadone L100 | | | | | | | 2.5 |
| LO097 | 2.5 | 2.5 | 2.5 | | 2.5 | 2.5 | |
| Acusol 445RD | | | | | | | |
| powder | | | | | | | |
| Sodium | 0.21 | 0.21 | 0.21 | 0.21 | 0.21 | 0.21 | 0.21 |
| Aluminate | | | | | | | |
| Dense ash | 3 | 3 | | | | 3 | 3 |
| (sodium | | | | | | | |
| carbonate) | | | | | | | |
| Light Ash | | | | | | | |
| (sodium | | | | | | | |
| carbonate) | | | | | | | |
| Sodium | | | | | 4 | | |
| Gluconate | | | | | | | |
| Sulfate | | | | | | | |
| Acusol 810A | | | | | | | |
| Propylene | | | | | 5.89 | | |
| Glycol | | | | | | | |
| Sodium Citrate | | | | | 7 | | |
| MGDA | | | | | 0.7 | | |
| Caustic:water | 63.3:36.7 | 63.8:36.2 | 64.0:36.0 | 64.3:35.7 | 47.0:53.0 | 63.8:36.2 | 63.8:36.2 |
| wt-ratio | | | | | | | |
| Total | 100 | 100 | 100 | 100 | 100 | 100 | 100 |

TABLE 6

| | Test Compositions | | | | | | |
|---|---|---|---|---|---|---|---|
| | 7 | 8 | 9 | 10 | 11 | 12 | 13 |
| Observa- | Solid | At least 3 layers | Hard solid | Overall | Ash settled | Soft solids. | Homogenous |
| tions | slightly soft. | separation. | underneath | softer solid | down and | Acusol 810A | solid except |
| | The ashes | Bottom a solid | small layer | with some | the viscosity | precipitated | the ashes at |
| | settled down | layer with gel- | of soft solid. | hase | was 56 cP, | out. | bottom, |
| | at bottom. | particles mixture. | Indicates | separation. | solidification | | viscosity 88 cp. |
| | | Top layer white | some phase | Light ash | with part of ash, | | |
| | | high viscosity | separation. | settling at | no obvious | | |
| | | oil-like. | | bottom. | phase separation. | | |
| | | Mid layer | | | | | |
| | | relatively thin. | | | | | |

TABLE 7

| | Test Compositions | | | | | | | |
|---|---|---|---|---|---|---|---|---|
| | 14 | 15 | 16 | 17 | 18 | 19 | 20 | 21 |
| Observa- | Turned to gel | Homogeneous | Some processing | Homogeneous | PSO | Unable to | No observations | Modification |
| tions | with addition | mixture with | concerns | solution. | powder | solidify | recorded | of mix order |
| | of glycerin | good viscosity. | with Acusol | Acusol 448 | able to | due to too low | | from |
| | but could be | Solidified | 448 (foaming, | powder | dissolve | caustic:water | | composition |
| | reversed with | into nice | exotherm, | did not fully | into | ratio. | | 25 did not |
| | temperature. | case | vaporizing). | dissolve, | solution. | Addition | | impact |
| | Too high of | | Good viscosity | | Low | of ash did not | | solidification |
| | viscosity and | | and solid | | viscosity | help | | |
| | not a smooth | | | | at 155 F. | solidification. | | |
| | surface | | | | | | | |
| | on the solid. | | | | | | | |

### Example 4

Additional testing was completed assessing solidification using the concentrated caustic solution for a pilot 5 gallon mixing for solid blocks. The testing to increase in size of the compositions was completed in the laboratory as summarized in Table 8 and the solidification observations are summarized in Table 9.

TABLE 8

| | Test Compositions | | | |
|---|---|---|---|---|
| Component | 2-1 | 2-2 | 2-3 | 2-4 |
| 72% NaOH Solution | 74.9 | 75.69 | 75.19 | 74.9 |
| Crude glycerin (85%) | 6 | 6 | 6 | 6 |
| Hexylene glycol | 0.1 | 0.1 | 0.1 | 0.1 |
| PSO | 5.74 | | 9.8 | 5.74 |
| PSO Powder | | 4.4 | | |
| 25R2 | 0.75 | 1 | 1 | 0.75 |
| Surfadone L100 | | | | 2.5 |
| SURFONIC LD-097 | 2.5 | 2.5 | 2.5 | |
| Acusol 448 | 6.8 | 10.1 | | 6.8 |
| Acusol 448 Powder | | | 5.2 | |
| Sodium Aluminate | 0.21 | 0.21 | 0.21 | 0.21 |
| Dense ash (sodium | 3 | | | 3 |
| carbonate) | | | | |
| Caustic:water wt-ratio | 63.8:36.2 | 64.7:35.3 | 64.8:35.2 | 63.8:36.2 |
| Total | 100 | 100 | 100 | 100 |

TABLE 9

| Test | | | | |
|---|---|---|---|---|
| Compositions | 1-Feb | 2-Feb | 3-Feb | 4-Feb |
| Observations | In situ | Insitu | Insitu | Not measured |
| | solidification | solidification | solidification | at scale up |
| | for cast solid | for case solid | for case solid | quantity |
| | controlled at | controlled at | controlled at a | |
| | temerature of | temperature of | temperature of | |
| | about 155 F. and | about 152 F. and | about 153 F. and | |
| | final viscosity | final viscosity | final viscosity | |
| | of about 4000 cP, | of about 960 cP | of about 850 cP | |
| | density about | density about | density about | |
| | 1.63 g/mL | 1.56 g/mL | 1.56 g/mL. The | |
| | | | composition | |
| | | | solidified quickly | |
| | | | with Acusol 448 | |
| | | | dispersed (not | |
| | | | dissolved) | |

The testing shows variations in viscosity impacted by components in the solid compositions. In some embodiments the increased Acusol 448 concentration increased viscosity. In some embodiments the targeted viscosity during the pouring of the composition into a container (often referred to as a packout step) is between about 1000-4000 cP for effective packout.

### Example 5

Additional testing was completed showing the dispensing rate of solid compositions. The testing was run at 20 psi and both 100° F. and 140° F. The test was set up to dispense solid product for 1 second every minute while measuring the mass of the capsule. The evaluated solids included an Inline caustic detergent having a 77.9:22.1 caustic:water ratio, compared to Composition 2-1 (an all liquid polymer formulation), Composition 2-2 (a Dry PSO formulation), and Composition 2-3 (a Dry Acusol formulation).

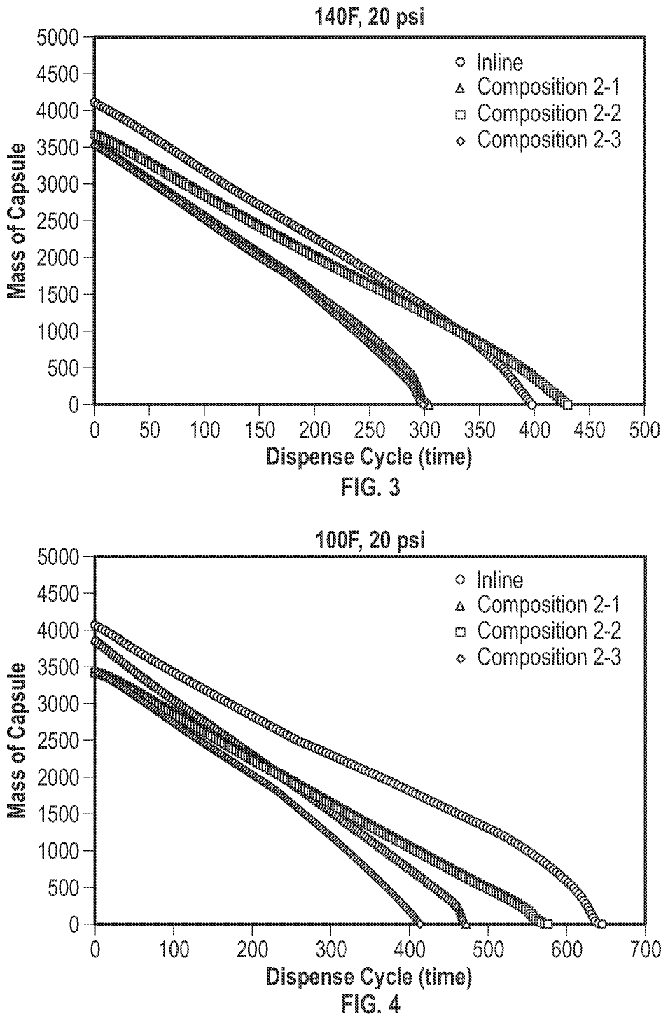

As shown in FIG. 3 and FIG. 4, the dispensing rate (slope of graphs showing mass over time) are within about 20% +/− of the inline caustic detergent. As shown in the figures the results show that the solid compositions are able to match the dispense rate of an inline product having increased caustic concentration and increased caustic:water wt-ratio. The dispensing rate is based on measurement of the dispense cycle (time) and the mass of the solid.

### Example 6

Additional testing was completed to assess the dimensional stability of the solid compositions evaluated in Example 5 (2-1, 2-2, 2-3) prepared as described according to the description herein. The measurement were obtained using a penetrometer reading that measures how far a needle penetrates the solid, after the solid sits for a certain amount of time (0, 2, 4, 6, 8 weeks and 24 hours at ambient conditions). The testing up to 8 weeks took place in a stability chamber as described and then removed from the controlled temperature chamber and placed under ambient conditions for 24 hours to assess whether any softening or swelling was reversible. The measurement assesses whether any softening of the solid takes place at the evaluated time points and conditions. The capsules were stored at different temperatures and a ‘cycle’ includes varying between 80° F. and 105° F. and 65%and 30% Relative Humidity. The conclusion is that the cast solid is softer than the inline caustic control, however this did not negatively affect the stability or dispense rates, due to the strength of the solid compositions. These results are shown in FIG. 6 (Composition 2-1), FIG. 8 (Composition 2-2), and FIG. 10 (Composition 2-3).

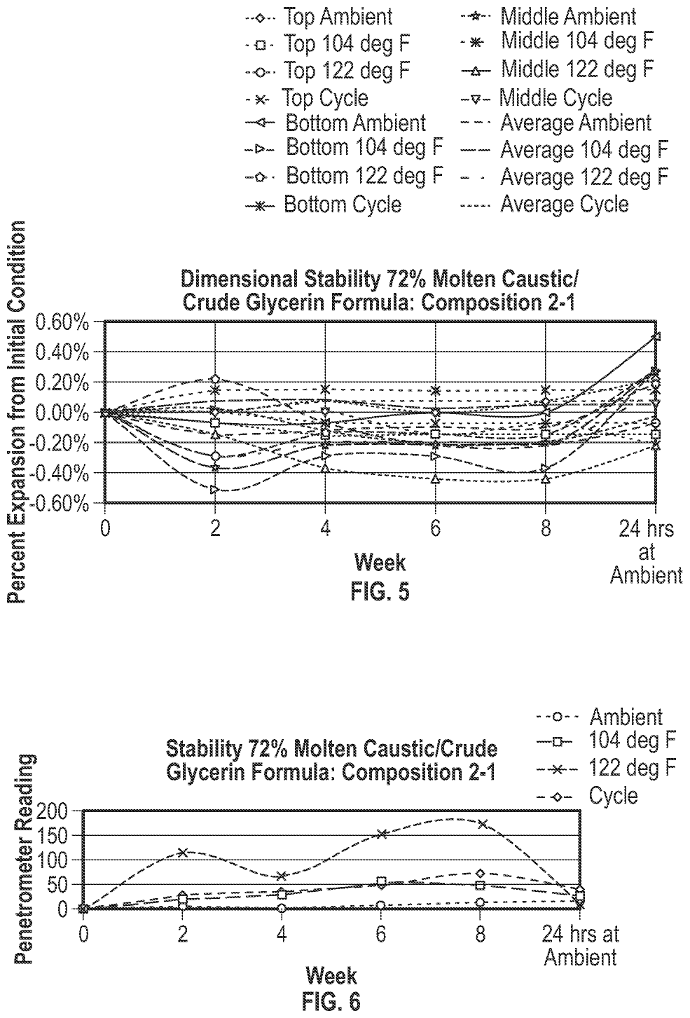

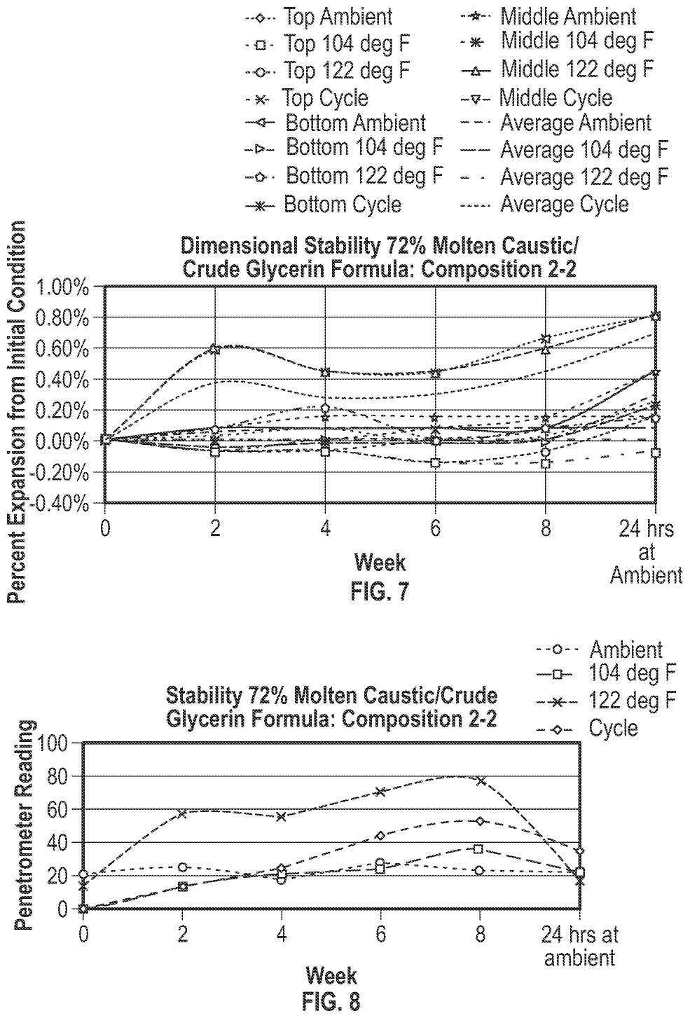

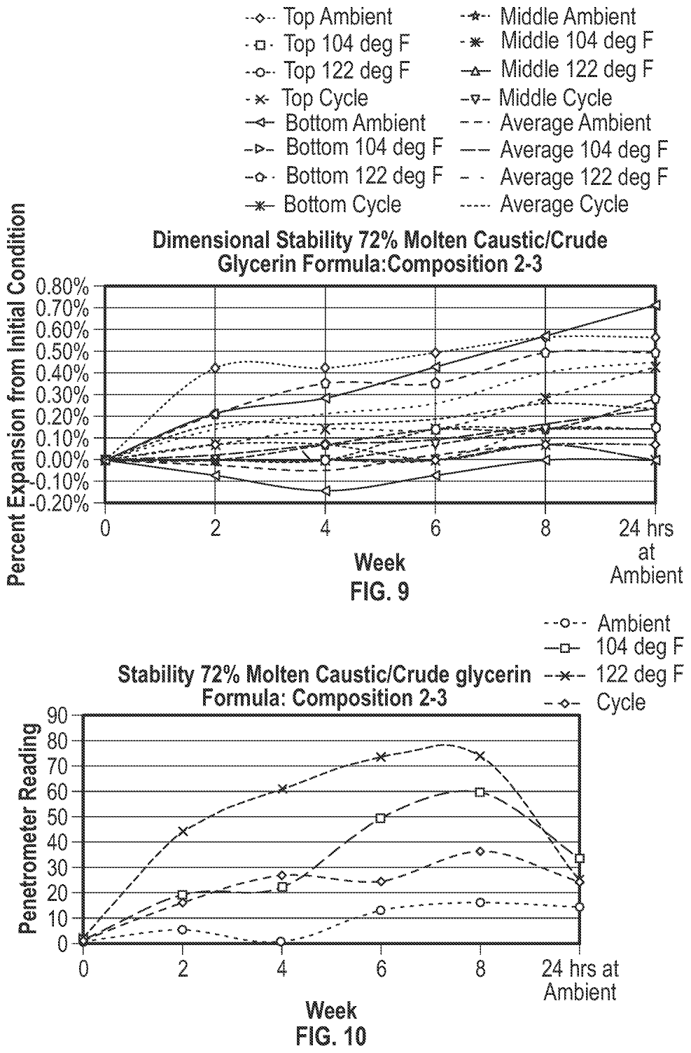

The results are shown in FIG. 5 (Composition 2-1), FIG. 7 (Composition 2-2), and FIG. 9 (Composition 2-3) wherein less than +/−0.6% change in expansion of the solids were observed by visual assessment over 8 weeks by testing expansion at the top, middle, and bottom (including average result), with measurements taken every 2 weeks for 8 weeks. In addition a measurement 24 hours after pouring is taken, which is shown at the far right (24 hrs at ambient). The results shown that the solids are stable even at low caustic:water ratios in the solid compositions. The variability of +/−0.6% change is approaching a 0% change (expansion or shrinking) indicating a stable solid that is suitable for packaging and dispensing in various conditions.

### Example 7

10-cycle testing was conducted with the solid compositions in Table 10 to evaluate use of the compositions in a warewashing machine as a detergent composition. The testing was performed in a Hobart AM-15 automated warewash machine, that goes through a 50 second wash at ˜160F and then a 10 second rinse at ˜180F. Ten wash cycles were completed, dosing the experimental detergent at 1000 ppm and a “hot point food soil” (beef stew, tomato soup, butter) at 2000 ppm. Water with a hardness of 5 grains per gallon (gpg) was used for testing. The ware tested were ceramic tiles. Prior to each cycle, the ceramic tiles were coated with a cream of chicken soup and whole milk mixture and heated in an oven ˜160F for 4 minutes.

The compositions 3-1, 3-2, and 3-3 were compared to commercially available Control having a greater concentration of caustic in the composition (including a caustic:water ratio of 77.9:22.1) which is increased caustic and increased caustic: water ratio than the evaluated compositions.

TABLE 10

| | Test Compositions | | |
|---|---|---|---|
| Component | 3-3 | 3-4 | 3-5 |
| 72% NaOH Solution | 80.6 | 74.9 | 74.9 |
| Crude glycerin | 6 | 6 | 6 |
| (85%) | | | |
| Hexylene glycol | 0.1 | 0.1 | 0.1 |
| PSO | | 5.74 | 5.74 |
| PSO Powder | 4.39 | | |
| 25R2 | 1 | 0.75 | 0.75 |
| Surfadone L100 | | | 2.5 |
| SURFONIC LD-097 | 2.5 | 2.5 | |
| Acusol 448 | | 6.8 | 6.8 |
| Acusol 448 Powder | 5.2 | | |
| Sodium Aluminate | 0.21 | 0.21 | 0.21 |
| Dense ash (sodium | | 3 | 3 |
| carbonate) | | | |
| Caustic:water wt- | 69.6:30.4 | 63.8:36.2 | 63.8:36.2 |
| ratio | | | |
| Total | 100 | 100 | 100 |

The results are shown in Table 11 where an improved L-value, a-value and b-value were measured by a Mach5 color instrument. The “L” value is a measure of brightness from 0-100, where larger values are whiter. The “a” value is a measure of red to green, where positive is more red and negative is more green. The “b” value is a measure of yellow to blue, where positive is more yellow and negative is more blue. Protein removal tests are run using a stain with a blue dye that shows residual protein after cleaning. A more negative “b” value means that more protein is left on the tile, indicating less cleaning efficacy. The more positive the “b” value, the cleaner the tile.

TABLE 11

| | Whiteness Evaluation - Averages | | |
|---|---|---|---|
| Formulations | L | A | b |
| Control-Inline | 80.44286 | 0.528571 | −14.1143 |
| 3-3 | 85.3875 | −0.2375 | −5.7375 |
| 3-4 | 85.9875 | −0.4125 | −5.4 |
| 3-5 | 82.125 | 0.175 | −10.5875 |

The results are surprising in that the compositions 3-3, 3-4, and 3-5 having lower caustic content (including caustic:water ratio) and therefore less alkaline compositions are able to provide efficient removal of soils. As a further benefit of the decrease in caustic there is further reduction in scaling in the less alkaline environment.

### Example 8

Additional performance results for compositions including a chelant were conducted according to the methods described in Example 7. The compositions are shown in Table 12 for making the solid composition.

TABLE 12

| | Test | Test |
|---|---|---|
| Component | Composition 4-1 | Composition 4-2 |
| 72% NaOH Solution | 70.9 | 73.6 |
| Crude glycerin (85%) | 6 | 6 |
| Hexylene glycol | 0.1 | 0.1 |
| PSO | 5.74 | |
| PSO Powder | | 2.75 |
| 25R2 | 0.75 | 0.75 |
| SURFONIC LD-097 | 2.5 | 2.5 |
| Acusol 448 | 6.8 | |
| Acusol 448 Powder | | 4 |
| Sodium Aluminate | 0.21 | 0.21 |
| MGDA | 7 | 10.09 |
| Caustic:water wt-ratio | 62.7:37.3 | 68.2:31.8 |
| Total | 100 | 100 |

The results of the 10-cycle testing at 5 gpg, 2000 ppm food soil, varying the detergent concentrations (as shown in Table 13) and tiles coated with milk and chicken soup mixture and heated 4 minutes between cycles. The blueness is the “b” value measured by the Mach5, where a more negative measurement indicates more protein residue after cleaning and less cleaning efficacy.

TABLE 13

| | | 4-1 | 4-1 |
|---|---|---|---|
| Formulations | Control | 1000 ppm | 600 ppm |
| Average blueness | −19.35 | −5.0 | −11.15 |

Then the results of a 50-cycle testing at harsher conditions of 17 gpg, 4000 ppm food soil, and at 1000 ppm detergent concentrations (as shown in Table 14) and tiles coated with milk and chicken soup mixture and heated 4 minutes between cycles. The results show a significant improvement in cleaning with the evaluated composition compared to control, including with a decrease in concentration to 600 ppm.

TABLE 14

| | | 4-1 |
|---|---|---|
| Formulations | Control | 1000 ppm |
| Average blueness | −7.8 | −4.65 |

TABLE 15

| | | 4-2 |
|---|---|---|
| Formulations | Control | 1000 ppm |
| Average blueness | −6.3 | +1.3 |

Again, the results in Table 15 show a significant improvement in cleaning with the evaluated composition compared to control. Beneficially, the solid compositions having a lower caustic:water ratio allow formulation space for additional performance enhancing components, including chelants such as aminocarboxylates.

### Example 9

The inclusion of chelants described in the prior example were also analyzed to further demonstrate solidification benefits of the binding effects of alkoxide-formed in addition to the binding of the chelant. The various solids evaluated with only the caustic:water and chelant evaluated are shown in Table 16.

TABLE 16

| | Composition | | Composition | | Composition | | Composition | | Composition | | Composition | |
|---|---|---|---|---|---|---|---|---|---|---|---|---|
| | 1 | | 2 | | 3 | | 4 | | 5 | | 6 | |
| RM | wt % | wt(g) | wt % | wt(g) | wt % | wt(g) | wt % | wt(g) | wt % | wt(g) | wt % | wt(g) |
| Water | 35 | 105 | 31.5 | 94.5 | 31.5 | 94.5 | 28 | 84 | 40 | 120 | 45 | 135 |
| Caustic | 65 | 195 | 58.5 | 175.5 | 59 | 175.5 | 72 | 216 | 60 | 180 | 55 | 165 |
| Bead | | | | | | | | | | | | |
| Glycerin | | | 10 | 30 | | | | | | | | |
| MGDA | | | | | 10 | 30 | | | | | | |

| NaOH:H₂O | 65:35 | 65:35 | | 65:35 | | 72:28 | | 60:40 | | 55:45 | |
|---|---|---|---|---|---|---|---|---|---|---|---|
| ratio | | | | | | | | | | | |
| | | Composition | | Composition | | Composition | | Composition | | Composition | |
| | | 7 | | 8 | | 9 | | 10 | | 11 | |
| | RM | wt % | wt(g) | wt % | wt(g) | wt % | wt(g) | wt % | wt(g) | wt % | wt(g) |
| | Water | 50 | 150 | 31.5 | 94.5 | 28 | 84 | 24.5 | 73.5 | 36 | 108 |
| | Caustic | 50 | 150 | 58.5 | 175.5 | 52 | 156 | 45.5 | 136.5 | 54 | 162 |
| | Bead | | | | | | | | | | |
| | Glycerin | | | | | 10 | 30 | 15 | 45 | 5 | 15 |
| | MGDA | | | 5 | 15 | 10 | 30 | 15 | 45 | 5 | 15 |

| | | | | | |
|---|---|---|---|---|---|
| NaOH:H₂O | 50:50 | 65:35 | 65:35 | 65:35 | 60:40 |
| ratio | | | | | |

The hardness of the solids was observed visually on a scale as summarized below form Soft to Hard: 7, 6, 5, 11, 1, 2, 10, 9, 8, 3, 4. The results can be summarized lower the caustic:water ratio below 69% caustic to water ratio (where a complete caustic monohydrate formation takes place), the solid becomes softer and may look wetter due to the mixture of caustic monohydrate and caustic dihydrates. When the polyol glycerin is added into the solid-forming composition the caustic: water ratio is lowered below the 69:31 ratio mentioned above, with a dry looking solid formed, demonstrating the benefit of the compositions and methods described herein. Still further, the incorporation of the chelant further increase the hardness of solids formed where a solid at as low as a 60:40 caustic to water ratio is achieved.

### Example 10

Quantification of the alkalinity and water in solid compositions described herein were conducted. Caustic:water weight ratios in solid compositions can be analytically confirmed by an acid/base titration using HCI that determines active alkalinity along with analytical calculation for water content with differential scanning calorimetry (DSC) scans in combination with literature values for the hydration states and melting points of sodium hydroxide to back-calculate the water percentage based on the weight of sample and latent heat of fusion to provide a quantitative measurement for water content.

Solid composition analyzed is shown in Table 17. Theoretical values are indicated as Th in the Table and shown as a percentage of the composition (Th %) for both water and sodium hydroxide.

TABLE 17
Formula 5

| | | | Water | NaOH | NaOH:H2O | Exp. | Exp. | Exp. |
|---|---|---|---|---|---|---|---|---|
| Description | Wt. % | Wt. (g) | (Th %) | (Th %) | (Th %) | water | NaOH | NaOH:H2O |
| NaOH (50%) | 39.7% | 198.5 | 19.85% | 19.85% | | | | |
| liquid | | | | | | | | |
| Caustic Bead | 31.2% | 156 | | 31.2% | | | | |
| Crude Glycerin | 6% | 30 | 0.60% | | | | | |
| PSO | 5.74% | 28.7 | 3.16% | | | | | |
| Acusol 448 | 6.80% | 34 | 3.40% | | | | | |
| Pluronic 25R2 | 0.75% | 3.75 | | | | | | |
| Sodium | 0.21% | 1.05 | 0.12% | | | | | |
| Aluminate | | | | | | | | |
| Hexylene | 0.10% | 0.5 | | | | | | |
| Glycol | | | | | | | | |
| SURFONIC | 2.50% | 12.5 | | | | | | |
| LD-097 | | | | | | | | |
| MGDA | 7% | 35 | 0.91% | | | | | |
| Caustic | | | 1.13% | −2.5% | | | | |
| neutralization | | | | | | | | |
| correction from | | | | | | | | |
| acidic polymers | | | | | | | | |
| | 100% | | 29.16% | 48.55% | 62.48% | 31% | 49.53% | 61.51% |

The analytical method for determining concentration of active alkalinity is by titration of a 1%solution of known volume to pH 8.3 with an acidic standard (used 0.1 M HCl). The analytical method for determining concentration of total alkalinity is by titration of a 1% solution with known volume to pH 4.0 with an acidic standard (used 0.1 M HCl).

The analytical methods for determining water content in the solid composition included the use of a high temperature oven. To determine the water content, a known sample of solid material's mass was recorded in a low humidity controlled environment. The sample was heated overnight in a low-humidity, high convection oven or a vacuum oven overnight with a temperature of at least 150° C. Then the final mass was recorded, making sure that no water was being absorbed from the environment due to the hygroscopic nature of the sample. Then all weight loss was assumed to be water evaporating from the sample to get a percent water.

As shown in Table 17 the difference between the calculated HaOH:H₂O (theoretical %) and the experimental or calculated NaOH:H₂O was 0.39% confirming the accuracy of the calculations as verified by the quantification. The measure of error for the method is acceptable as <1%. These differences between the theoretical and experimental NaOH confirms that the titration methods are accurate for determining caustic.

It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. Any reference to accompanying drawings which form a part hereof, are shown, by way of illustration only. It is understood that other embodiments may be utilized and structural changes may be made without departing from the scope of the present disclosure. All publications discussed and/or referenced herein are incorporated herein in their entirety.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

What is claimed is:

1. A solid composition comprising:
an alkali metal hydroxide;
a polyol; and
water;
wherein the solid has a weight ratio of alkali metal hydroxide to water from about 60:40 to less than about 70:30,
wherein the water is from any component of the solid composition and/or water added as a raw material; and
wherein the solid is a contiguous solid, powder or granule.

2. The solid composition of claim 1, wherein the alkali metal hydroxide is sodium hydroxide, potassium hydroxide, lithium hydroxide, derivatives thereof, or combinations thereof.

3. The solid composition of claim 1, wherein the polyol is a C1-C22 alcohol, a glycol, or derivative thereof, or a combination thereof.

4. The solid composition of claim 1, wherein the polyol is a diol, triol, and/or polyol containing more than 3 hydroxyl groups.

5. The solid composition of claim 1, wherein the polyol is a glycol and/or glycerin.

6. The solid composition of claim 5, wherein the glycol is an aliphatic glycol comprising ethylene glycol, propylene glycol, hexylene glycol, 1,4-butanediol, or combinations thereof.

7. The solid composition of claim 1, further comprising detersive surfactant, water conditioning agent, chelating agent, hydrotrope, carbonate alkalinity source, anhydrous material to absorb excess water in the solid composition, or combinations thereof.

8. The solid composition of claim 7, wherein the detersive surfactant or surfactant combination comprises a first surfactant comprising a reverse EO/PO block copolymer of about 20-40% EO for protein soil defoaming, and a second surfactant that is a reverse EO/PO block copolymer of about 40% EO, an alkyl capped alcohol ethoxylate (preferably a butyl capped alcohol ethoxylate), or alkyl pyrrolidone (preferably a C8, or C10 alkyl pyrrolidone) for protein soil removal from wares.

9. The solid composition of claim 7, wherein the chelating agent is an aminocarboxylate and/or Sodium Gluconate.

10. The solid composition of claim 9, wherein the aminocarboxylate chelating agent is selected from the group consisting of MGDA, NTA, EDTA, DTPA, and TTHA.

11. The solid composition of claim 1, wherein the solid is a dimensionally stable cast solid.

12. A method of making a solid composition comprising:
combining a concentrated alkali metal hydroxide and a polyol to generate in situ a solid composition,
wherein the concentrated alkali metal hydroxide comprises greater than 50% (actives basis) liquid alkali metal hydroxide, and
wherein the solid has a weight ratio of alkali metal hydroxide to water from about 60:40 to less than about 70:30,
wherein the water is from any component of the solid composition and/or water added as a raw material, and
wherein the solid is a cast solid, tablet, or block, and wherein the solid is a dimensionally stable solid.

13. The method of claim 12, wherein the method further comprises an initial step of concentrating an alkali metal hydroxide by either (i) concentrating a liquid alkali metal hydroxide having 50% (actives basis) or less alkali metal hydroxide by evaporation, or (ii) by dissolving a solid alkali metal hydroxide in a liquid alkali metal hydroxide having 50% (actives basis) or less, to provide the concentrated alkali metal hydroxide.

14. The method of claim 12, wherein the step of combining concentrated alkali metal hydroxide and polyol provides the components into a capsule or container to form the solid cast, tablet, block, or powder, and preferably where the components are added to the capsule or container at a sufficiently high viscosity.

15. The method of claim 12, wherein the concentrated alkali metal hydroxide is from about 69% to about 74% (actives basis) liquid alkali metal hydroxide, or from about 70% to about 73% (actives basis) liquid alkali metal hydroxide.

16. The method of claim 12, wherein the method does not use a chiller for solidification.

17. The method of claim 12, wherein the solid composition is formed between about 1 minute and about 3 hours, or between about 1 minute and about 20 minutes.

18. A method of using a solid composition comprising:
generating a use solution of the solid composition according to claim 1 or the composition made by the methods of claim 12;
contacting an article or surface in need of cleaning, disinfecting, and/or sanitizing with the use solution; and
cleaning, disinfecting, and/or sanitizing the article or surface.

19. The method of claim 18, wherein the solid composition does not slough during dispensing to generate the use solution.

20. The method of claim 18, wherein the solid composition comprises less than about 70:30 alkali metal hydroxide to water ratio and provides improved cleaning performance in comparison to compositions comprising additional alkali metal hydroxide.
